(19) United States
(12) Patent Application Publication
(10) Pub. No.: US 2026/0066049 A1
(43) Pub. Date: Mar. 5, 2026

(54) HIGH-RESOLUTION AND NON-INVASIVE FETAL SEQUENCING

(71) Applicants: The General Hospital Corporation, Boston, MA (US); The Broad Institute, Inc., Cambridge, MA (US)

(72) Inventors: Michael E. Talkowski, Boston, MA (US); Harrison Brand, Boston, MA (US); Christopher Whelan, Jamaica Plain, MA (US)

(21) Appl. No.: 19/107,188

(22) PCT Filed: Aug. 30, 2023
(86) PCT No.: PCT/US2023/031556

§ 371 Date: Feb. 27, 2025

Related U.S. Application Data
(60) Provisional application No. 63/402,379, filed on Aug. 30, 2022.

(51) Int. Cl. G16B 30/10; C12Q 1/48; C12Q 1/6806; C12Q 1/686; C12Q 1/6869; C12Q 1/6876; G16B 5/00; G16B 20/10; G16B 20/20; G16B 40/20; G16H 10/40; G16H 10/60; G16H 50/30

(52) U.S. Cl. CPC G16B 30/10; C12Q 1/48; C12Q 1/6806; C12Q 1/686; C12Q 1/6869; C12Q 1/6876; C12Y 207/07; G16B 5/00; G16B 20/10; G16B 20/20; G16B 40/20; G16H 10/40; G16H 10/60; G16H 50/30; C12Q 2600/156

(57) ABSTRACT

Provided herein are computer-implemented methods for assigning maternal or fetal origin to one or more genetic variants in cell free DNA (cfDNA) from a sample from a pregnant mammal, preferably a pregnant human, using a probabilistic model for assigning maternal or fetal origin to genetic variants in DNA from a sample obtained from a pregnant mammal, wherein the model assigns maternal or fetal origin based on a combination of fetal fraction and DNA fragment size.

### CLAIM OF PRIORITY

This application claims the benefit of U.S. Provisional Application Ser. No. 63/402,379, filed on Aug. 30, 2022. The entire contents of the foregoing are incorporated herein by reference.

### FEDERALLY SPONSORED RESEARCH OR DEVELOPMENT

This invention was made with Government support under Grant No. HD081256 awarded by the National Institutes of Health. The Government has certain rights in the invention.

### TECHNICAL FIELD

Provided herein are methods (e.g., computer-implemented methods) for assigning maternal or fetal origin to one or more genetic variants in cell free DNA (cfDNA) from a sample from a pregnant mammal, preferably a pregnant human, using a probabilistic model for assigning maternal or fetal origin to genetic variants in DNA from a sample obtained from a pregnant mammal, wherein the model assigns maternal or fetal origin based on a combination of fetal fraction and DNA fragment size.

### BACKGROUND

Early genetic diagnosis of a fetus can guide clinical management, predict outcomes, and provide a basis for precision medicine.¹⁻³

### SUMMARY

Provided herein are computer-implemented methods that can be used for assigning maternal or fetal origin to one or more genetic variants in cell free DNA (cfDNA) from a sample from a pregnant mammal, preferably a pregnant human. The methods comprise (a) accessing, from memory, a probabilistic model for assigning maternal or fetal origin to genetic variants in DNA from a sample obtained from a pregnant mammal, wherein the model assigns maternal or fetal origin based on a combination of fetal fraction and/or DNA fragment size and other sequencing features; (b) inputting, into the model, a set of values representing one or more genetic variants detected in the cfDNA from a peripheral blood sample from a pregnant mammal, wherein the values include empirically determined sequence information, e.g., ratio of different bases in the read, and DNA fragment size information, e.g, a rank sum statistic, for each genetic variant; and (c) assigning, using the model, maternal or fetal origin for the one or more genetic variants.

In some embodiments, the genetic variants comprise single nucleotide variants (SNVs), indels, and/or copy number variations (CNVs).

In some embodiments, an initial set of values representing the one or more genetic variants is obtained by a method comprising: aligning raw sequencing reads derived from the cfDNA to a reference genome sequence; transforming the raw sequencing reads into consensus reads; realigning the consensus reads to the reference genome sequence, thereby producing a set of aligned consensus reads; identifying consensus reads that differ from the reference genome; assigning consensus reads that differ from the reference genome as alternate alleles and assigning consensus reads that match the reference genome as reference alleles, and determining a fragment size rank sum statistic representing the distribution of the estimated fragment sizes of reads supporting the reference allele as compared to the distribution of the fragment sizes of reads supporting the alternate allele, thereby obtaining an initial set of values representing sequence identity and DNA fragment size rank sum statistic for one or more genetic variants.

In some embodiments, each of the raw sequencing reads comprises a unique molecular identifier (UMI); and the method comprises transforming the raw sequencing reads into a single consensus read for each UMI.

In some embodiments, the methods further comprise selecting a set of candidate variants before step (b), by a method comprising: accessing, from memory, a machine learning classifier, optionally a random forest based model, wherein the machine learning classifier is trained using a set of predetermined filter criteria and a subset of sites present in the sample or in reference samples to identify potential false positive (FP) sites; inputting, into the machine learning classifier, the initial set of variants; and filtering, using the trained machine learning classifier to remove a set of variants enriched for false positive (FP) sites, thereby selecting a set of candidate variants from the initial set.

In some embodiments, the probabilistic model uses k-means or a Bayesian Mixture Model that simultaneously estimates fetal fraction and assigns fetal or maternal origin for each variant site in the set. In some embodiments, the Bayesian Mixture Model is a Bayesian Gaussian Mixture Model constrained over variant allele fraction and fragment size, e.g., a fragment size rank sum statistic.

In some embodiments, the fetal fraction of the sample is modeled as a latent variable (f) and mean of the variant allele fraction distribution is set for each component based on f.

In some embodiments, the fetal fraction is estimated based on a reference fetal fraction determined based on clusters derived from VAF across sites.

In some embodiments, the methods further comprise outputting a list of one or more genetic variants identified as having fetal origin and/or one or more genetic variants identified as having maternal origin.

In some embodiments, the methods further comprise comparing the genetic variants to a database that comprises a list of genetic variants and information regarding variants that are potentially medically relevant to the fetus or mother; identifying variants present in the fetus or the mother that are potentially medically relevant; and outputting a list of the one or more genetic variants identified as having fetal origin and/or one or more genetic variants identified as having maternal origin that potentially medically relevant.

In some embodiments, the methods further comprise the methods can further include recommending further testing based on the presence of variants that are potentially medically relevant.

In some embodiments, the further testing comprises amniocentesis or chorionic villus sampling (CVS); further monitoring of the fetus via ultrasonography; or genetic testing of the mother.

In some embodiments, the methods further comprise using high throughput sequencing on cfDNA extracted from a single sample of peripheral blood from the mother, optionally wherein exome capture is performed before the sequencing. The present methods need not (and typically do not) use paternal blood samples or sequences (e.g., for benchmarking or any other purpose), and optionally do not use a separate maternal only sample (e.g., for benchmarking or any other purpose); the methods can include, but do not have to, determining maternal genotype from leukocytes as described herein, and in some embodiments the methods are solely performed using cfDNA from a single sample of plasma from the mother. Thus the present methods can be performed using a single sample, rather than requiring independent samples from maternal and paternal genome, or to normalize to a reference panel.

In some embodiments, adaptors with common PCR primer sequences and unique molecular identifiers (UMIs) are attached to the cfDNA, and PCR amplification is performed before the sequencing.

In some embodiments, the methods further comprise enriching the sample for fetal DNA, optionally by contacting the cfDNA with a plurality of oligonucleotides that bind to portions of the fetal genome, optionally comprising fetal protein-coding genes or other regions of the fetal genome that may be relevant to clinical interpretation or variant identification.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-C. Workflow for non-invasive fetal exome screening with NIFS. FIG. 1A shows the process for extracting cell-free DNA (cfDNA) from maternal plasma followed by exome capture. We generated 51 libraries across gestational ages and calculated the fetal fraction (fetal cfDNA/total cfDNA) in each sample. FIG. 1B highlights the novel variant detection methods developed to account for fetal fraction and the corresponding unique allelic fractions at each site depending on the maternal and fetal genotype combinations present in cfDNA. Each cluster represents a unique maternal/fetal genotype combination, and clusters are colored by genotypes generated from direct exome sequencing (ES) of maternal and fetal DNA. These data highlight the merging of clusters at heterozygous sites in the mother at lower coverage and fetal fraction, and the clearly resolved de novo and paternally derived variants (fetal 0/1; maternal 0/0) irrespective of fetal fraction. FIG. 1C shows application of NIFS to 14 cases referred for invasive testing and representative variants of clinical interest in Table 4, including a likely pathogenic splice variant in COL2A1 (NC_000012.12:g.47982610C>T) in a fetus with micrognathia consistent with Stickler syndrome, a 4 MB pathogenic deletion on chromosome 7 in a fetus with multiple congenital anomalies (NC_000007.14:g.155368937-159327017del), and maternal carrier screening detecting a PAH risk variant (NC_000012.12:g.102866632C>T) associated with phenylketonuria (PKU). All variants were orthogonally validated (Table 2).

FIG. 2. Exemplary Workflow. An exemplary workflow in which data processing is divided into three stages. In the Alignment and Preprocessing stage, raw sequencing reads derived from exome based sequencing (ES) of cfDNA are aligned to the reference genome, grouped by unique molecular identifiers (UMIs), and transformed into a single consensus read for each UMI. Consensus reads are then realigned to the reference and base quality scores are recalibrated, producing a set of aligned consensus reads that are ready for downstream variant calling and analysis. Next, in the Variant Filtering and Genotyping stage of the workflow, candidate variants sites are identified using Mutect2; variants are filtered using a set of hard filters and a random-forest based model trained on a subset of sites present in that sample; and a Bayesian Mixture Model is used to simultaneously estimate the fetal fraction and assign fetal and maternal genotypes to each site. Finally, in Variant Interpretation all passing variants are annotated and evaluated to produce a list of clinically relevant variants for interpretation.

FIGS. 3A-C. The “Unfiltered Variant Detection”, “Filtered Variant Detection”, “Overall Genotyping Performance”, “Predicted Paternal or de novo Variant Detection” and “Genotyping Accuracy for Variants Heterozygous in the Mother” evaluations are plotted against fetal fraction. Theoretical sensitivity and detection of non-maternal variants is strong across fetal fractions, while genotyping accuracy, especially for variants which are heterozygous in the mother, is worse at lower fetal fractions. Sensitivity: TP/(TP+FN); PPV: TP/(TP+FP); Genotype Accuracy: Percent of maternal heterozygous variants assigned the correct fetal genotype.

FIG. 4. We were able to separate male and female cases through assessment of sequencing coverage on chrY. Examination of the number of intervals on chrY with mapped sequencing reads allowed us to detect a confirmed male vanishing twin with a female fetus (Table 13), which had read coverage over a much larger proportion of chrY than other samples from pregnancies with female fetuses. Investigating predicted chrY copy state was less accurate, but we did find an extreme case where the mother had received a stem cell transplant from a male donor and therefore had coverage on chrY six times higher than expected. In addition, lower normalized chrY depth distinguished a twin pregnancy with discordant sexes. We also highlight a single sample with lower-than expected fetal fraction, which exhibited depressed coverage on chrY, and a pregnancy with two XX fetuses, which clustered with other samples that had pregnancies with a single XX fetus.

FIG. 5. Variant Allele Fraction Graph. Histogram of observed variant allele fractions (proportion of reads supporting the alternate allele) as plotted for all autosomal sites observed in a sample with 38% fetal fraction at 268x coverage. The peaks of the distribution are shown with their assignment to maternal or fetal genome genotypes based on their mean and variance according to the fetal fraction and coverage.

FIG. 6. Non-Invasive Fetal Sequencing (NIFS) overview. We collect plasma (mixture of fetal and maternal DNA; Step 1) and then extract DNA using (Step 2). UMIs are attached and exome sequence is captured (Step 3). Bioinformatic analysis is then performed to generate accurate fetal and maternal genotypes exome-wide (Step 4). Interpretation is the performed using best clinical practice (Step 5).

FIGS. 7A-B. A) Shows the process for extracting cell-free DNA (cfDNA) from maternal plasma followed by exome capture. We are able to extract both plasma, which consists of fetal and maternal DNA, and DNA from leukocytes, which is solely maternal DNA. The unique maternal DNA from leukocytes can used for independent variant validation and maternal carrier screening. B) UMIs are attached, exomes are captured, sequencing libraires are built and sequencing is performed on an Illumina machine.

FIGS. 8. Fragment Size as a Genotyping Feature. Each plot shows the distribution of fragment sizes of reads which could be definitively assigned a fetal or maternal origin. Reads were assigned an origin if they overlapped a variant site which allowed one allele to be unambiguously associated with either the fetus or the mother based on the genotypes derived from maternal and cord blood or amniocentesis WES. For example, if the maternal genome was homozygous reference and the fetal genome was heterozygous, all reads supporting the alternate allele could be assigned a fetal origin. In each sample fetal-derived fragments are enriched for small fragments and depleted of larger fragments relative to maternally-derived fragments.

FIG. 9. Filtering and Genotyping Performance. Cell free fetal DNA is enriched for short fragments compared to maternal and we devised a rank sum test to show these deviations. A lower rank sum statistics indicates an increased number of shorter fragments indicating that variant is more likely to be of fetal origin. This information correlates well with the VAF predictions and we use both of these metrics in our genotyping method.

FIG. 10. Variant Calling Workflow. Overview of variant calling processing involving initial variant detection with mutect that can optionally be filtered by maternal genotype if generated from leukocytes as described in FIG. 7. Variants are initially filtered with a machine learning technique to remove false positive and the genotyped with a Bayesian Gaussian mixture model as described below.

FIG. 11. Model Diagram of the graphical model used for genotype assignment. The model is a Bayesian Gaussian mixture model defined over the variant allele fraction and fragment size statistic (computed by the InsertSizeRankSumTest) for each site, where the means of the variant allele fraction components are constrained by a latent variable estimating the fetal fraction. Information of model is shown in the table below.

FIG. 12. Exemplary Data Processing Workflow.

FIG. 13 is a schematic diagram of an example computer system.

### DETAILED DESCRIPTION

Non-invasive prenatal screening (NIPS) has been transformative for the discovery of aneuploidies. However, recent systematic benchmarking studies have shown that this current state-of-the-art low-resolution approach captures only a small fraction of the pathogenic and likely pathogenic variants in fetuses harboring a structural anomaly detected on ultrasound (˜26% diagnostic yield from aneuploidy screening alone), whereas a much greater diagnostic yield can be achieved by sequencing and analysis of nucleotide changes and copy number variants (CNVs) that alter protein coding sequences in the human genome (42-48%) (Lowther et al. 2020, Biorxiv; in press AJHG). Several recent studies have shown that a targeted approach on a small panel of genes or CNVs known to be relevant to prenatal diagnosis can be targeted in cfDNA⁴⁻¹⁵, but at present comprehensive genetic screening of the fetal coding sequence or whole-genome during pregnancy still requires an invasive procedure that carries inherent risks for the mother and fetus, such as amniocentesis.

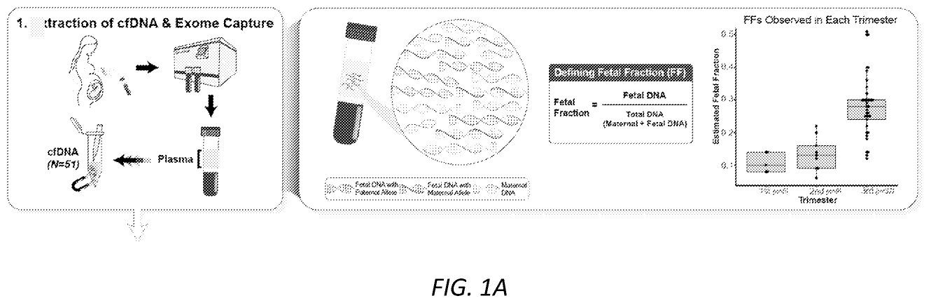

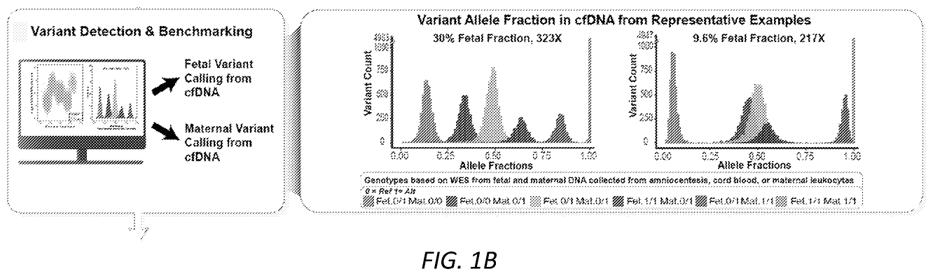

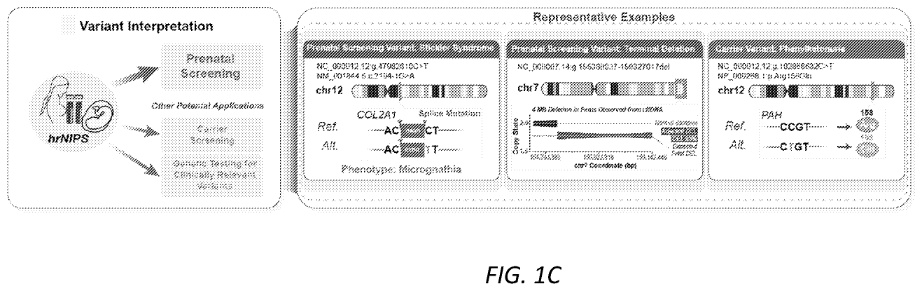

Demonstrated herein is an integrated molecular and computational process to facilitate scalable and non-invasive fetal sequencing (NIFS) to discover and annotate individual nucleotide sequence changes and CNVs from circulating cell-free DNA (cfDNA) extracted from maternal plasma. This high-resolution NIFS approach can survey all >22,000 protein coding genes in the fetal exome (NIFS-E) that encompasses virtually all interpretable pathogenic variation in fetal diagnostic testing (see Lowther et al. benchmarking from invasive studies of fetal structural anomaly (FSA) cases) or be applied for complete genome sequencing (NIFS-G). Here, we focus on NIFS-E as a novel approach to simultaneously provide a non-invasive survey of the complete fetal exome as well as routine maternal carrier screen during pregnancy without the need for a paternal sample (FIGS. 1A-C). The success of this method has implications for the displacement of current standard-of-care microarray and exome sequencing from invasive procedures for prenatal genetic diagnosis, as well as the enterprises of neonatal sequencing, newborn screening, and maternal carrier testing.

We sequenced samples from 51 pregnancies, including 37 samples from third trimester pregnancies for methods development and 14 samples that were collected during the first (n=5) or second (n=9) trimesters, consistent with current applications of NIPS, and observed fetal fractions ranging from 6% to 51%. Applying NIFS, we captured and sequenced 22,995 genes from the fetal genome (18,049 protein coding genes). We detected and genotyped single nucleotide variants (SNVs) and indels using a custom pipeline that applied a Bayesian Gaussian mixture model to account for fetal fraction while incorporating approaches such as fragment length and sequencing features of cfDNA into our model. We further leveraged these features and read-depth ratios for CNV discovery from the cfDNA samples. At much lower sequencing cost than clinical whole genome sequencing, the NIFS method generated a median of 203-fold exome-wide read coverage. We further benchmarked NIFS against 11 samples with germline exome sequencing from cord blood or amniocentesis and captured 99.7% of all 298,576 SNV sites detectable from standard exome sequencing while maintaining a median sensitivity of 93.0% and precision of 93.4% after precise genotyping of all variants. Fetal sex was accurately inferred for all samples. Importantly, there was minimal impact of fetal fraction from our methods on de novo or paternally inherited variants, suggesting the capacity to screen for de novo variation in fetuses very early during pregnancy.

As a validation experiment, we assessed the clinical utility of NIFS across 14 pregnancies referred for routine genetic testing and detected 100% of variants of interest identified from current standard-of-care clinical testing, including a pathogenic de novo CNV in a fetus with multiple congenital anomalies, a likely pathogenic splice variant in COL2A1 in a fetus with micrognathia, and a homozygous pathogenic indel in CFTR likely to cause cystic fibrosis. This NIFS approach accessed both maternal and fetal cfDNA, which also provided high-sensitivity discovery for maternal SNVs (98.3% sensitivity against standard exome sequencing) and carrier screening that yielded at least one reportable variant in 57.1% of mothers evaluated, which comported with previous estimates^{16,17}.

The potential utility of NIFS in prenatal screening is broad. The method provides nucleotide resolution screening to displace the currently low-resolution NIPS approach. It could also provide a rapid reflex test for pregnancies with ultrasound abnormalities prior to the need for an invasive procedure. Variant discovery can be utilized and re-interpreted neonatally when exome sequencing is currently warranted^{18,19}, which could dramatically reduce the time to diagnosis for many conditions. We also identified a variant that may modulate risk for later onset conditions of relevance (i.e. a BRCA2 variant associated with breast cancer risk) and such variants can be interpreted for parental risk based on criteria from the American College of Medical Genetics based on reportable secondary findings using existing guidelines in the current standard-of-care^{20,21}. Indeed, the NIFS approach provides comparable genetic data to current invasive prenatal exome sequencing and requires the same detailed guidelines and procedures for interpretation and the appropriate return of results²². These analyses indicate that the complete fetal exome is accessible using new molecular and analytic approaches such as NIFS from the same maternal plasma samples that are already routinely collected for lower resolution fetal screening.

### Non-Invasive Fetal Sequencing (NIFS) Methodology

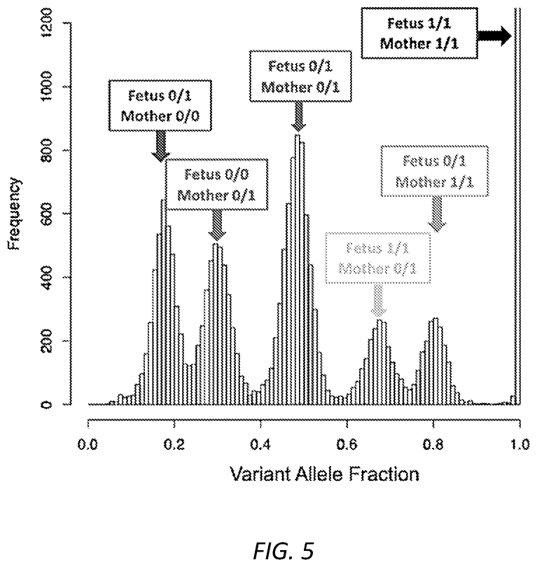

The variant allele fraction can be used to inform predictions about small variant genotypes, as genetic variants present in the cfDNA are a mixture of fetal and maternal fragments. Reads supporting a variant depend on the maternal and fetal genotypes as well as fetal fraction; these patterns help predict genotype for both mother and fetus. FIG. 5 shows an exemplary graph of VAF plotted against frequency annotated to show the component assigned using the Bayesian Gaussian Mixture Model described herein. Fetal fraction decreases cause cluster means to shift. Sequencing depth can also affect the outcome, as lower coverage causes higher variance within clusters; low coverage and low fetal fraction can challenge the ability to distinguish fetal genotypes based solely on VAF in sites where the mother is heterozygous.

In the present methods, fetal variants are uniquely detectable with high sensitivity and specificity using the NIFS analytic pipeline as described herein, which takes fragment size into account as well.

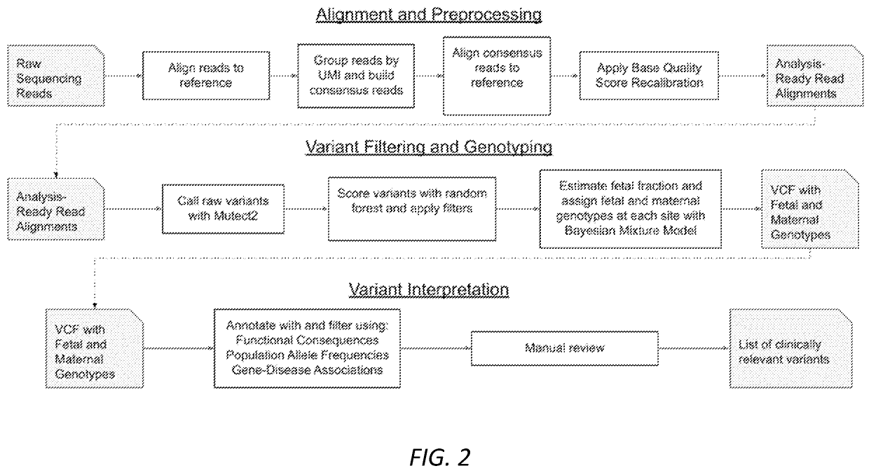

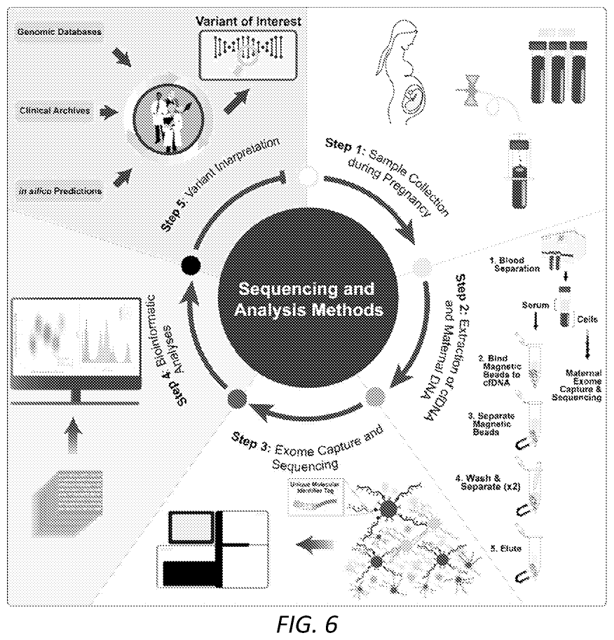

FIG. 6 provides a schematic overview of an exemplary NIFS workflow; an exemplary workflow is shown in FIG. 2. As shown, the methods are performed on samples collected from a pregnant woman (Step 1, although the present methods can be performed on samples previously collected and the methods need not require a sample collection step). In Step 2, cfDNA (and optionally maternal DNA, e.g., obtained from leukocytes) are extracted from the sample. Next, exome capture is optionally performed, and the cfDNA (and optionally maternal DNA) are sequenced in Step 3. Bioinformatic analysis of the sample is performed in Step 4, and variant interpretation in Step 5.

Samples (i.e., peripheral blood samples) can be collected using methods known in the art. In some embodiments, 5-40 ml, e.g., 20 ml, is collected via blood draw in pregnant subjects. The present methods can be used in mammals, e.g., humans or non-human veterinary subjects. The present methods need not (and typically do not) use paternal blood samples or sequences (e.g., for benchmarking or any other purpose), and optionally do not use a separate maternal only sample (e.g., for benchmarking or any other purpose); the methods can include, but do not have to, determining maternal genotype from leukocytes as described herein, and in some embodiments the methods are solely performed using cfDNA from a single sample of plasma from the mother. Thus the present methods can be performed using a single sample, rather than requiring independent samples from maternal and paternal genome, or to normalize to a reference panel.

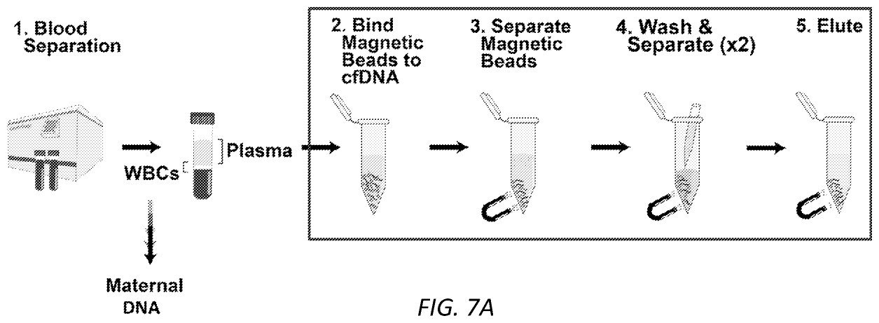

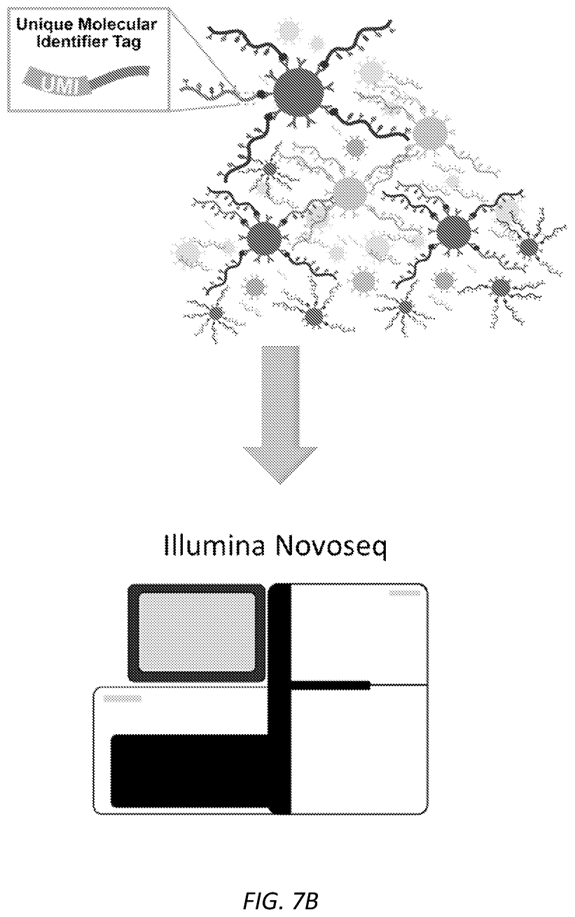

After the sample is subjected to plasma separation, cfDNA is extracted from the plasma (representing a mixture of fetal and maternal cfDNA); DNA can optionally also be extracted from leukocytes (only maternal DNA that can be used for validation). DNA extraction can be performed using methods known in the art, e.g., as shown in FIG. 7A. An exemplary method is described below in the section title Library Creation Methods; briefly, the plasma is mixed with magnetic beads that bind to cfDNA, then a magnetic field is applied to concentrate the beads, which are then washed, separated, and eluted. Other methods for isolating cfDNA are known in the art and can be used, e.g., spin-column based, manual magnetic bead-based, and automatic magnetic bead-based methods (see, e.g., Polatoglou et al., Diagnostics (Basel). 2022 October; 12(10): 2550; Bronkhorst et al., Tumour Biol. 2020 April; 42(4):1010428320916314; Bronkhorst et al., Tumour Biol. 2019 August; 41(8):1010428319866369; Michelson et al., Mitochondrion. 2023 July; 71:26-39; Streleckiene et al., Biopreserv Biobank. 2019 December; 17(6):553-561; Solassol et al., Clin Chem Lab Med. 2018 Aug. 28; 56(9):e243-e246). A number of kits are available for isolation, including QIAamp Circulating Nucleic Acid Kit (QiaM, 55114 Qiagen GmbH, Hilden, Germany), NucleoSpin Plasma XS (Macherey-Nagel 740900.50, high-sensitivity protocol MNaS, Macherey-Nagel GmbH, Duren, Germany), QIAmp MinElute ccfDNA Mini Kit (QiaS, 55204, Qiagen GmbH, Hilden, Germany), cfPure Cell-Free DNA Extraction Kit (BChM, K5011610-BC, BioChain Inc., Newark, CA, USA), MagMAX Cell-Free DNA Isolation Kit (TFiM, A29319, Thermo Fisher Scientific, Waltham, MA, USA) and automated methods include the MagNA Pure 24 Total NA Isolation Kit (RocA, 07658036001, Roche Diagnostics GmbH, Penzberg, Germany), NextPrep-Mag™ cfDNA Automated Isolation Kit (PerkinElmer), and the cfNA ss 2000 protocol on the MagNA Pure 24 System (Roche Diagnostics).

Preferably, adaptors with common primer sequences and unique molecular identifiers (UMIs) are attached to the DNA to maximize sequence coverage, and PCR is used to amplify the library. The methods can then include an optional step of enriching the sample for fetal DNA, e.g., by contacting the cfDNA with a plurality of oligonucleotides that bind to portions of fetal protein-coding genes, e.g., a TWIST target panel (Alliance Clinical Research Exome), optionally targeting all 22,995 genes from the fetal genome (or the 18,049 protein coding genes, or a subset thereof) or a subset thereof, or other regions of the fetal genome that may be relevant to clinical interpretation or variant identification, or the methods can including sequencing all of the nucleotides in the genome without exome capture (this method is referred to herein as NIFS, genome; NIFS-G). High throughput/next generation sequencing methods are then used to sequence the UMI-tagged DNA (either from the total cfDNA population, e.g., genomic DNA, or exome-enriched DNA), preferably to an average depth of sequencing of about 100×, 150×, or 200×. A filtered sequencing depth (i.e., after the UMIs are used to filter out the relevant reads) of at least 200, 250, or 300× in the first and second trimester, and at least 100× (but more preferably 200, 250, or 300×) in the third trimester, is preferred. See FIG. 7B.

Sequencing can be performed using methods known in the art, including automated Sanger sequencing (e.g., using an ABI 3730x1 genome analyzer), pyrosequencing on a solid support (e.g., using 454 sequencing, Roche), sequencing-by-synthesis with reversible terminations (e.g., using an ILLUMINA® Genome Analyzer), sequencing-by-ligation (ABI SOLiD®) or sequencing-by-synthesis with virtual terminators (HELISCOPE®); Moleculo sequencing (see Voskoboynik et al. eLife 2013 2:e00569 and U.S. patent application Ser. No. 13/608,778, filed Sep. 10, 2012); DNA nanoball sequencing; single molecule real time (SMRT) sequencing; Nanopore DNA sequencing; sequencing by hybridization; sequencing with mass spectrometry; and microfluidic Sanger sequencing. Exemplary next generation sequencing methods known to those of skill in the art include Massively parallel signature sequencing (MPSS), Polony sequencing, pyrosequencing (454), Illumina (Solexa) sequencing by synthesis, SOLiD sequencing by ligation, Ion semiconductor sequencing (Ion Torrent sequencing), DNA nanoball sequencing, chain termination sequencing (Sanger sequencing), heliscope single molecule sequencing, single molecule real time (SMRT) sequencing (Pacific Biosciences); flow-based sequencing (e.g., Ultima sequencing) and nanopore sequencing such as is described at world wide website nanoporetech.com.

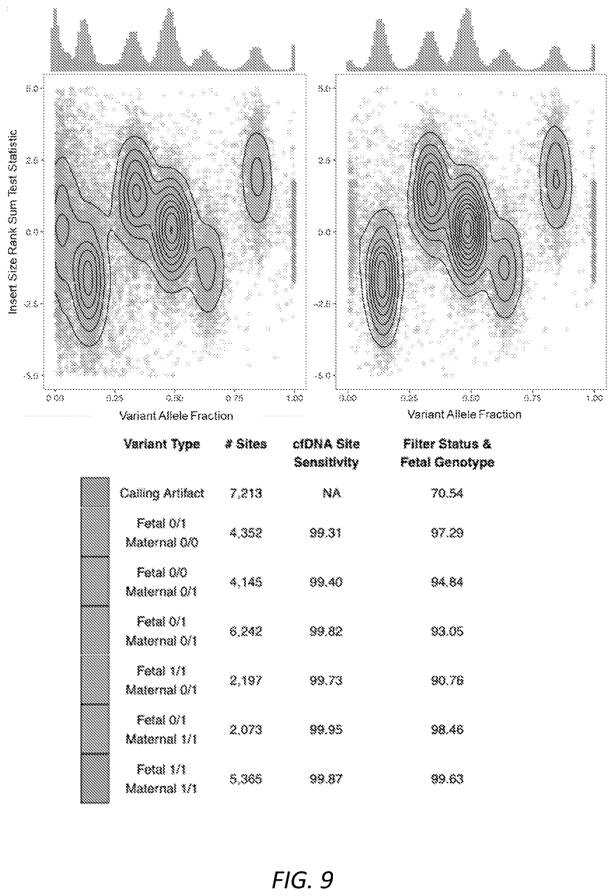

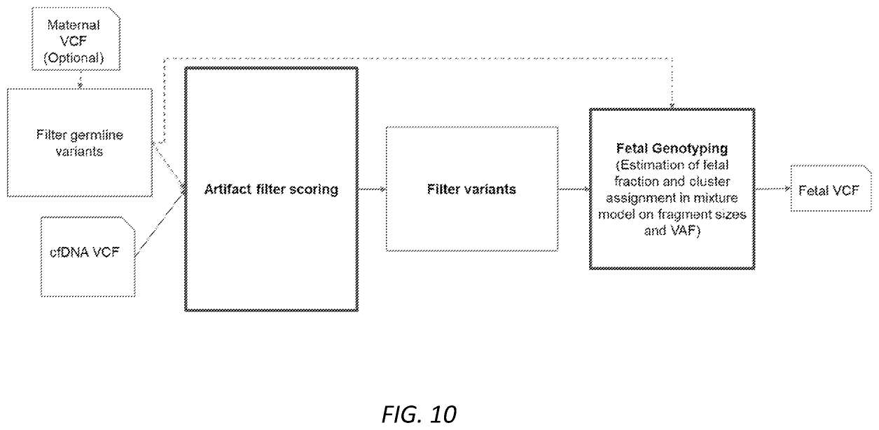

Novel bioinformatics analysis methods are then used to detect and identify variants from the sequencing data, to discover short variants (e.g., single nucleotide variants (SNVs) and indels) and CNVs. In general, the data processing methods can be divided into three stages: alignment and preprocessing; variant filtering and genotyping; and variant interpretation. See, e.g., FIG. 9.

In the Alignment and Preprocessing stage, raw sequencing reads derived from the cfDNA are aligned to a reference genome, grouped by UMI, and transformed into a single consensus read for each UML. Consensus reads are then realigned to the reference and base quality scores are optionally recalibrated to improve read quality, producing a set of aligned consensus reads that are ready for downstream variant calling and analysis. Maternal genotyping can optionally be performed, and then the maternal genome or a database can be used to filter germline variants. The genotyping data is optionally in Variant Call Format (VCF), a file format is used to encode genetic variant sites and genotypes.

Next, in the Variant Filtering and Genotyping stage of the workflow, candidate variant sites are first identified by comparison to a reference genome (e.g., GRCh38 using Mutect2). The candidate variants can be filtered to remove potential false positive (FP) sites, e.g., using a set of hard filters and a machine learning classifier, e.g., a random forest-based model, support vector machine (SVM), or Neural Net, which is trained on a subset of sites present in that sample. A probabilistic model is then applied to estimate fetal fraction and assign fetal and/or maternal genotypes to all variant sites observed in the cfDNA sequencing data; for example, a k-means or Bayesian Mixture Model can be used, e.g., to simultaneously estimate the fetal fraction and assign fetal and maternal genotypes to each site.

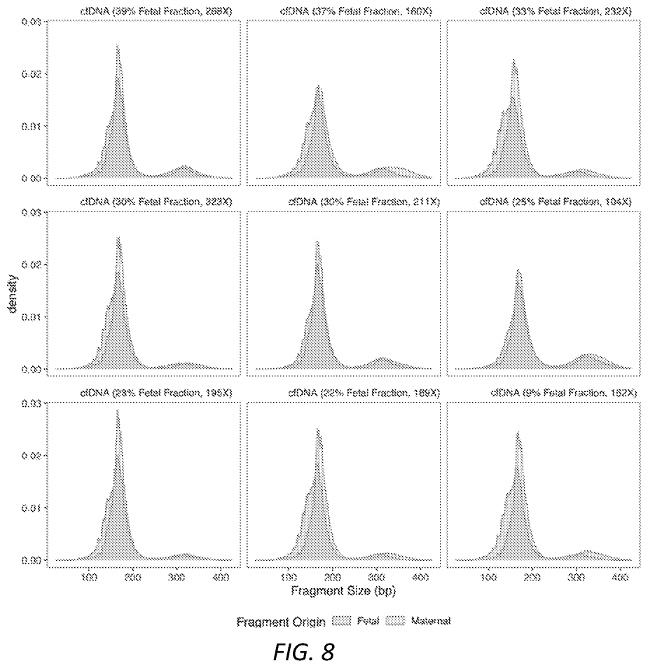

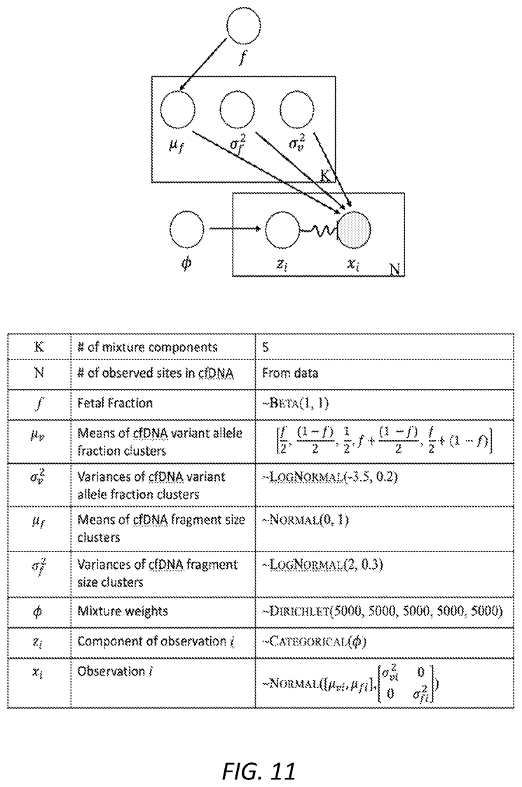

In some embodiments, the probabilistic model simultaneously estimates fetal fraction and assigns fetal and maternal genotypes to all variant sites observed in the cfDNA sequencing data using a constrained 2D Bayesian Gaussian Mixture Model with five components, with each component representing a different combination of maternal and fetal genotypes for an autosomal variant. The combinations are defined over two dimensions: the variant allele fraction (VAF) and a fragment size rank sum statistic that summarizes the difference between fragments sizes of reads supporting the reference and alternate alleles, e.g., as described herein (e.g., in the section Variant Detection of cfDNA with Mutect2); see, e.g., FIG. 8. The centers of the cfDNA VAF clusters are determined by fetal fraction (FF). The model (shown in FIG. 11) can be fit using stochastic variational inference (e.g., using Pyro).

Table A shows the five components used in the exemplary Bayesian Gaussian Mixture Model.

TABLE A
Variant Allele Fractions

| Fetal Genotype | Maternal Genotype | Expected VAF |
|---|---|---|
| 0/1 | 0/0 | ff/2 |
| 0/0 | 0/1 | (1 − ff)/2 |
| 0/1 | 0/1 | 0.5 |
| 1/1 | 0/1 | ff + (1 − ff)/2 |
| 0/1 | 1/1 | ff/2 + (1 − ff) |
| 1/1 | 1/1 | 1 |

ff = fetal fraction;
VAF = Variant Allele Fraction: reads variant allele/total reads

As shown in FIG. 9, the incorporation of fragment size and variant allele fraction (VAF) into the probabilistic model allows for accurate assignment of origin (maternal or fetal or both), e.g., based on assignment to one of the five components shown above.

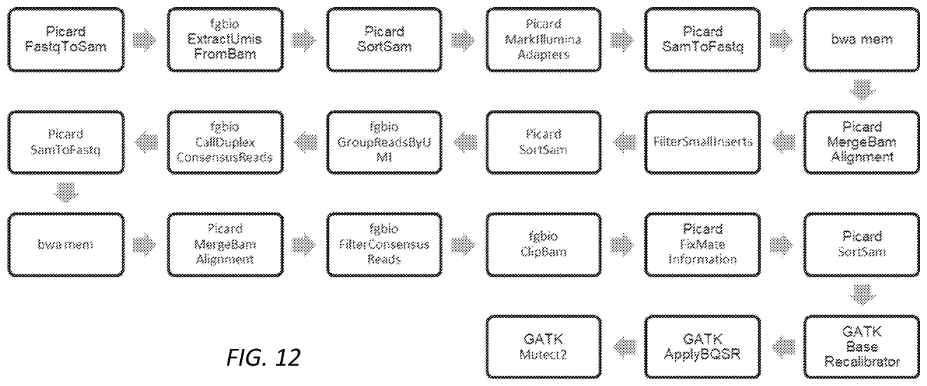

Finally, in Variant Interpretation all passing variants are annotated and evaluated to produce a list of clinically relevant variants for interpretation. Annotation can be performed by reference to one or more databases, for example, the variants can be annotated with genic and functional consequences (e.g., based on RefSeq⁴), allele frequency (e.g., based on gnomAD v2.1.1 and gnomAD v3.0), Rare Exome Variant Ensemble Learner (REVEL)¹⁶ scores that predict the deleteriousness of each nucleotide change in the genome, ClinVar¹⁷ annotations (updated 2023 Apr. 30), and per gene disease information such as inheritance type (e.g. recessive, e.g., e.g., based on the Online Mendelian Inheritance in Man database (OMIM, version 2022-07-08¹⁸). The variants can be further filtered, e.g., included if they had an allele frequency of <5 or were not reported in gnomAD v2.1.1 and gnomAD v3.0⁶, or excluded if determined likely benign or benign/likely benign in ClinVar, or synonymous variants. See, e.g., FIG. 12 for an exemplary data processing workflow.

The results can then be used to output a list from each sample for further review, preferably including all ClinVar annotated Pathogenic/Likely Pathogenic variants, all frameshift/stopgain variants, all predicted splice variants with a Splice Al score¹⁹>0.95, all non-frameshift variants >15 amino acids; and all non-synonymous variants with a REVEL score >0.7. The list can be shared, e.g., with health care providers, or with the mother.

Based on the presence of variants in the fetus that have potential to have a deleterious effect on the health of the fetus or mother (e.g., pathogenic or likely pathogenic variants, including those associated with negative health conditions or outcomes), the methods can further include recommending further testing, e.g., invasive testing such as amniocentesis or chorionic villus sampling (CVS), and/or further monitoring via ultrasonography.

Based on the presence of variants in the fetus that have potential to be deleterious, the methods can further include recommending further testing, e.g., genetic testing to confirm the variants.

Standard computing devices and systems can be used and implemented to perform the methods described herein. Computing devices include various forms of digital computers, such as laptops, desktops, mobile devices, workstations, personal digital assistants, servers, blade servers, mainframes, and other appropriate computers. In some embodiments, the computing device is a mobile device, such as personal digital assistant, cellular telephone, smartphone, tablet, or other similar computing device. The components described herein, their connections and relationships, and their functions, are meant to be exemplary only, and are not meant to limit implementations of the inventions described and/or claimed in this document.

Computing devices typically include one or more of a processor, memory, a storage device, a high-speed interface connecting to memory and high-speed expansion ports, and a low-speed interface connecting to low speed bus and storage device. Each of the components are interconnected using various busses, and can be mounted on a common motherboard or in other manners as appropriate. The processor can process instructions for execution within the computing device, including instructions stored in the memory or on the storage device to display graphical information for a GUI on an external input/output device, such as a display coupled to a high-speed interface. In other implementations, multiple processors and/or multiple buses can be used, as appropriate, along with multiple memories and types of memory. Also, multiple computing devices can be connected, with each device providing portions of the operations (e.g., as a server bank, a group of blade servers, or a multi-processor system).

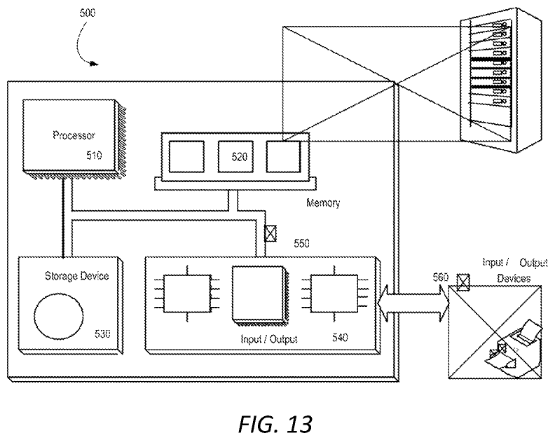

FIG. 13 shows an example computer system 500 that includes a processor 510, a memory 520, a storage device 530 and an input/output device 540. Each of the components 510, 520, 530 and 540 can be interconnected, for example, by a system bus 550. The processor 510 is capable of processing instructions for execution within the system 500. In some implementations, the processor 510 is a single-threaded processor, a multi-threaded processor, or another type of processor. The processor 510 is capable of processing instructions stored in the memory 520 or on the storage device 530. The memory 520 and the storage device 530 can store information within the system 500.

The input/output device 540 provides input/output operations for the system 500. In some implementations, the input/output device 540 can include one or more of a network interface device, for example, an Ethernet card, a serial communication device, for example, an RS-232 port, or a wireless interface device, for example, an 802.11 card, a 3G wireless modem, a 4G wireless modem, or a 5G wireless modem, or both. In some implementations, the input/output device can include driver devices configured to receive input data and send output data to other input/output devices, for example, keyboard, printer and display devices 560. In some implementations, mobile computing devices, mobile communication devices, and other devices can be used. In some embodiments, the present methods are performed using a device comprising a sequencing machine, e.g., an Illumina sequencer.

### EXAMPLES

The invention is further described in the following examples, which do not limit the scope of the invention described in the claims.

### Methods

The following methods were used in the Examples below.

### Sample Collection

Samples were collected from the Brigham and Women's Hospital (BWH) LIFECODES longitudinal biorepository²³ and the MAPing pregnancy study biorepository based out of the Center for Fetal Medicine at BWH (Tables 1-2). This resource was designed to facilitate research on prenatal screening and diagnosis and understanding of the genetic basis of fetal structural anomalies. We collected samples from any gestation period with initial technology development focusing on the third trimester, while the 14 samples harboring fetal anomalies were amassed primarily from first and second trimester (Tables 3, and 12). Women were enrolled at prenatal visits during any time of pregnancy and peripheral blood samples were collected in two Streck collection tubes (Streck, La Vista, NE) providing up to 20 mL of maternal blood.

51 total samples were collected from 49 singleton pregnancies, 1 dizygotic twin pregnancy, and 1 monozygotic twin pregnancy. The samples were collected across all 3 trimesters, and 11 samples had matched confirmation samples for obtaining benchmarking data using fetal DNA obtained from cord blood or amniocentesis.

### Library Creation Methods

Following sample collection, we separated cell free DNA (cfDNA) from maternal serum using Streck's recommended ‘Double Spin Protocol 2’. Precipitated maternal leukocytes were used to extract maternal genomic DNA from all samples. To perform extensive benchmarking of maternal variant discovery, we collected maternal germline DNA (gDNA) for 28 mothers and performed standard exome sequencing (ES) at the Broad Institute Genomics Platform (Table 3). We extracted DNA from the separated cfDNA portion of the serum with a NextPrep Mag cfDNA Isolation kit (Catalog #NOVA-3825-03). We then determined cfDNA fragment size and concentration via Tapestation (cfDNA tapes, Agilent Technologies) and QuBit (Broad Range DNA, Agilent Technologies), respectively. To convert cfDNA to sequenceable fragments, we used NEBNext® Ultra™ II DNA Library Prep Kit for Illumina® from New England Biolabs (NEB) according to the manufacturer's protocols with the following modifications: 1) NEB adapters and USER enzyme steps were replaced with direct ligation of xGen Stubby unique molecular identifier (UMI) adapters ordered from integrated DNA technologies (IDT); and 2) NEB primers were replaced with xGen dual index primer (IDT). After adapter ligation, PCR was then performed for 12 cycles. Following this initial PCR amplification, libraries were multiplexed into batches of up to 16 (up to 8 μg of total material) and exome capture was performed using the Alpha Broad Exome baits from TWIST Bioscience, targeting 194,202 exonic regions, under the IDT xGen Hybridization Protocol. In brief, multiplexed libraries were combined with Human Cot DNA and xGen blocking oligos and dehydrated prior to resuspension in hybridization buffer and baits. After four hours incubation, bait hybridized libraries were combined with buffer resuspended streptavidin beads and several washes were performed to remove any non-hybridized libraries, followed by 15 rounds of on-bead, post-capture PCR. PCR amplified libraries were purified using SPRI bead clean up, and exome libraries were analyzed with a tapestation (D1000, Agilent Technologies) and QuBit (Broad Range DNA, Agilent Technologies), prior to multiplexing and sequencing on an Illumina NovaSeq. We were able to obtain additional material for benchmarking analyses in a subset of the participants in the study, including fetal cord blood collected at delivery (n=7), paternal DNA (n=7), and in an additional four cases DNA was extracted from cultured cells derived from an amniocentesis (Table 3).

### Sequence Generation

cfDNA libraries were sequenced at the Broad Institute Genomics Platform in pooled, multiplex sequencing runs on an Illumina Novaseq S4 flowcell. Our multiplexing strategy sought to generate as many unique sequencing reads as possible while keeping the raw sequence duplication rates (without considering UMIs) under 75%. We note that at a depth of 200x, assuming a fetal fraction of 25% (the median fetal fraction observed across our samples), each target was expected to have mean coverage of approximately 50 reads of fetal origin. For eight samples (MGB038, MGB039, MGB40, MGB016, MGB043, MGB046, MGB047, and MGB048) sequencing was performed across two S4 flowcells and the raw sequencing reads were pooled and processed together.

### Data Processing Workflow Overview

An overview of an exemplary data processing workflow is given in FIG. 2. The workflow is divided into three main sections, which are described at a high level in this section and in greater detail hereinbelow. The first stage preprocessed raw sequencing reads, built consensus reads for each UMI found in the sequencing data set, and aligned those consensus reads to the reference genome. See “Alignment and Preprocessing of cfDNA Sequence Data” for a more detailed description of the methods and tools. In the next stage of processing, the somatic variant caller Mutect2 was used to generate candidate variant call sites from the aligned consensus reads (see section “Variant Detection in cfDNA”); a machine-learning based approach was then used to train a classifier for each sample's data set to filter variant sites that are likely to be artifacts (see “cfDNA Variant Filtering” section); and then a Bayesian Mixture Model was used to simultaneously estimate fetal fraction and assign fetal and maternal genotypes to each variant site (“cfDNA Genotyping”). Finally, a set of protocols was developed for annotating, interpreting, and curating variant sites to produce a list of potentially clinically relevant variants for each sample (“Variant Determination”).

Alignment and Preprocessing of cfDNA Sequence Data

The following pipeline was used to generate high quality GRCh38 aligned cram files for variant calling. First, UMIs were extracted from each read using the open source fgbio ExtractUmisFromBam (github.com/fulcrumgenomics/fgbio) from Fulcrum Genomics. Several subsequent steps were performed using the open-source Picard tool from the Broad Institute of MIT and Harvard (broadinstitute.github.io/picard/), including sorting the data by query name using Picard SortSam. Illumina adapters were identified and marked with Picard's MarklluminaAdapters. Reads were then converted to FASTQ with Picard's SamToFastq, aligned to the GRCh38 reference genome with the open source BWA-MEM aligner²⁴, and merged back into a BAM file with Picard's MergeBamAlignment. We then removed a small number of degenerate mapped fragments with mapped fragment length smaller than 19 bp with the PrintReads tool in the open source GATK²⁵ framework from the Broad Institute (gatk.broadinstitute.org). Reads were then grouped by UMI with the fgbio tool GroupReadsByUmi. We created consensus duplex reads with fgbio CallDuplexConsensusReads with parameters--error-rate-pre-umi45--error-rate-post-umi=30--min-input-base-quality=10--min-reads=0. Consensus reads were filtered with fgbio FilterConsensusReads with parameters--min-reads 0 0 0--max-read-error-rate 0.35--max-base-error-rate 0.3--min-base-quality 40--max-no-call-fraction 0.25 and then clipped with fgbio ClipBam using parameters--clipping-mode=Hard--clp-overlapping-reads=true. Mate information was fixed and the mate CIGAR tags were populated with Picard's FixMateInformation. Consensus reads were sorted by coordinate using Picard's SortSam. Finally, Base quality scores were recalibrated with the GATK BaseRecalibrator and ApplyBQSR tools. Metrics collection, as well as variant calling, filtering, and genotyping were then applied to the covered target intervals in the Twist Broad Custom exome kit (Twist Alliance Clinical Research Exome). A publicly available version of the covered targets data is available at: twistbioscience.com/resources/data-files/twist-alliance-clinical-research-exome-349-mb-bed-files.

Covered gene counts were calculated by intersecting this target interval list with the GRCh38 refSeq database²⁶ downloaded from the UCSC genome browser (NCBI Annotation Release 110).

### Coverage Analysis

We applied the Picard tool CollectHsSequencingMetrics to collect coverage statistics across the all-exome targets based on aligned consensus reads. In Table 3 we report, for each sample, the mean coverage across all target intervals and the fraction of target bases with at least 50x coverage by cfDNA sequencing reads (of mixed maternal and fetal origin). We also multiplied per-target mean coverage metrics for each sample by that sample's estimated fetal fraction to produce the percentage of exome target intervals with a mean estimated fetal read coverage of at least 8x and 1Ox, and report both values for each sample in Table 4. Finally, in samples with matched paternal gDNA ES, we report the median and inter-quartile range of the number of reads supporting paternal-only alleles in Table 7, “Genotyping Performance and Coverage Based on Paternal gDNA ES”. These values can be used to infer the distribution of the coverage of these sites by sequencing reads originating from the fetal genome, half of which are expected to support the paternal allele.

Variant Site Detection in cfDNA

We identified candidate variant sites using the open source software tool Mutect2²⁷ from the Broad Institute of MIT and Harvard with the parameter--max-mnp-distance 0, to split MNP variants into separate records, and the following parameters to generate annotations used in filtering: -G StandardAnnotation -G StandardHCAnnotation -A MappingQualityZero -A TandemRepeat -A CountNs. We generated an additional annotation to use in genotyping (see the section cfDNA Genotyping) by modifying GATK to add an InsertSizeRankSum annotation to each variant based on the fragment sizes of reads supporting the reference and alternate alleles at each site. To produce this annotation, the distribution of the estimated fragment sizes of reads supporting the reference allele was compared to the distribution of the fragment sizes of reads supporting the alternate allele using a Mann-Whitney U test (implemented by GATK's RankSumTest). The value of the annotation is the Z score of the U statistic. Fragment sizes were estimated for each read determined to be informative at each site by the Mutect2/GATK assembly-based calling engine based on the mapped insert size reported by BWA in the BAM file for each read pair and were adjusted to account for insertions and deletions reported in the CIGAR and mate CIGAR of the read.

cfDNA Variant Filtering

To remove potential false positive (FP) sites due to sequencing, library preparation, or alignment error, variant site filters were developed that included hard filtering rules and a random forest-based classifier that assigned a score to each variant site that reflected the likelihood that the site is a true positive (TP) variant. The filtering rules were:
1. Any sites in the cfDNA sample were filtered if Mutect2 identified more than one alternate allele and for which at least one of the alternate alleles was an indel.
2. A machine learning classifier (described in detail below) was applied to score variants and filter any variants with a score lower than a cutoff determined by assessing sensitivity to a gold standard set of common variants.
3. Indels that were likely recurrent sequencing errors but still passed our random forest filter were hard filtered based on a list of recurrent artifactual indel calls we observed in our data sets. To construct this list, we identified every indel site with an allele count of at least 5 in the subset of cfDNA samples from this study that did not have a matched cord blood or amniocentesis sample and were not among the samples that had been referred for genetic testing. From this resulting list we removed any sites that were present at any allele frequency in the gnomAD v3.1.2²⁸ database. The remaining sites were used to make a catalog, consisting of 969 indels, which were recurrent artifacts in our data. We applied a filter to remove any indels sites with a position and alternate allele that matched one of the sites in this catalog.
4. Any site that was confidently called by Mutect2 and in phase with a SNV site that did not pass one or more of the filters listed above was also filtered. Mutect2 calls certain sets of sites to be in phase with one another based on the number of reads which span more than one site in the set and support the same combination of alleles. Information is recorded in the phase set ID (PID) annotation for the variant. This filter catches clustered sets of sites that represent mapping errors when reads originate from other paralogous sequences in the genome that contain multiple paralog specific variants.

In addition to the filters listed above, two filters were applied after genotyping (see section cfDNA Genotyping) based on identifying variant sites with unexpectedly low counts of reads supporting the alternate allele.

The machine learning classifier described in step 2 above was built using a scheme based upon the principle of positive-unlabeled learning²⁹, in which only positive training labels are known with certainty in a training data set. We trained a new instance of this classifier for every sample, using only data from that sample. Reasoning that variant sites that are common in the population are likely to be real, we assigned initial positive labels to sites that are present in gnomAD v3²⁸ with a maximum sub-population frequency (as given by the AF_popmax annotation in the gnomAD data) of at least 0.1. All other sites were initially assigned a negative training label. We then trained a random forest with 800 estimators implemented by the scikit-learn package. After training the classifier we then scored each variant with the predicted probability of the site being a true positive according to the classifier. We then identified a cutoff for this score for PASS filter status using GATK's FilterVariantTranches tool, which finds optimal cutoffs that result in a requested estimated sensitivity based on a set of common SNPs and indels supplied as resources with the best practices pipeline. In our pipelines we requested sensitivities of --snp-tranche 99.5 and --indel-tranche 95.0. The following features, which were chosen to be independent of allele fraction and were either generated by Mutect2 or added based on the genomic context of the site's coordinates, were selected for assessment in the random forest:
Indel: Binary feature indicating whether the variant is a SNP or an indel
SOR: Strand-odds-ratio strand bias test statistic
MQ: Root mean squared mapping quality
MQRankSum: Mapping quality bias rank sum test
ReadPosRankSum: Read position bias rank sum test
BaseQRankSum: Test of base quality score bias for reference and alternate alleles
MPOS: Median distance of site from end of read
ECNT Number of events in the assembled haplotype containing the variant
NCount: Number of reads in the pileup with an N basecall (created in the formation of duplex consensus reads) at the variant site
DP: Depth at the variant site
SEGDUP: Binary features indicating whether the site lies within a segmental duplication
LCR: Binary features indicating whether the site lies within a low complexity region as defined by the LCR-hs38 resource provided by Li et al.³⁰
SIMPLEREP: Binary feature indicating whether the site lies within an annotated simple repeat
STR: Binary feature indicating whether GATK/Mutect classifies the site as falling within a short tandem repeat sequence.

It should be noted that the fact that a variant was observed in a repetitive genomic region (as annotated by the SEGDUP, LCR, SIMPLEREP, and STR annotations) was used as a feature for training in the classifier, rather than as a hard filter, with the goal of allowing the classifier to make confident calls in those regions of the genome.

cfDNA Genotyping and Estimation of Fetal Fraction

We developed a machine learning-based model that simultaneously estimates fetal fraction and assigns fetal and maternal genotypes to all variant sites observed in cfDNA sequencing data. Our model consists of a constrained Bayesian Gaussian Mixture Model with five components, with each component representing a different combination of maternal and fetal genotypes for an autosomal variant. The mixtures were defined over two dimensions: the variant allele fraction and the fragment size rank sum statistic summarizing the difference between fragments sizes of reads supporting the reference and alternate alleles, described in the section Variant Site Detection in cfDNA. We modeled the fetal fraction of the sample as a latent variable (f) and set the mean of the variant allele fraction distribution for each component based on it as follows: If we let 0/0 represent a homozygous reference genotype, 0/1 represent a heterozygous genotype, and 1/1 represent a homozygous alternate genotype, the components and their means are defined as: (“cluster 0”: fetal 0/1, maternal 0/0)f 2; (“cluster 1: fetal 0/0, maternal 0/1”) (1−f) 2; (“cluster 2: fetal 0/1, maternal 0/1”) 0.5; (“cluster 3: fetal 1/1, maternal 0/1”)f+(1−f) 2; (“cluster 4: fetal 0/1, maternal 1/1”) 1−(f/2). Each data dimension was modeled independently, i.e., the covariance matrix for each component was diagonal. Prior to running inference on the model's parameters, we removed a subset of sites that appeared to be outliers, including sites with non-passing filter status (as set by the filtering procedures described above in cfDNA Variant Filtering), sites with cfDNA VAF less than 0.025 or greater than 0.975, and sites with fragment size statistics that were missing, less than −4, or greater than 4. To further clean the data, we removed any sites that did not pass an outlier test for cfDNA VAF and fragment size statistics. The outlier test was implemented by fitting an IsolationForest outlier classifier from the sklearn.ensemble package to the data with a contamination parameter of 0.05. We defined the genotyping mixture model in Pyro³¹ and fit it to the data for each sample using stochastic variational inference. We used Pyro's AutoDelta guide functions to find the maximum aposteriori values for each parameter. To initialize the model, we first produced an initial estimate of the fetal fraction. We did this by identifying the location of the cluster of sites in the VAF distribution representing sites that are maternal homozygous variants and heterozygous in the fetus (“cluster 4”). We initialized the fetal fraction by computing the Gaussian kernel density estimate of all sites with VAF less than 0.975 and identifying the peak in the density with the largest value, corresponding to cluster 4, using the scipy.signal.argrelextrema function. To estimate the initialization value for the mean of the fragment size statistic distribution, we found the 500 sites with cfDNA VAF closest to the expected VAF for cluster 4 based on the estimated fetal fraction and used their median fragment size statistic value. Once the fragment size statistic distribution mean for the maternal homozygous variant/fetal heterozygous sites was estimated, we initialized the means of the other fragment size component distributions by multiplying this value times the vector [−1.0, 0.5, 0.0, −0.5, 1.0] to match the expected relative contributions of maternal vs. fetal reads observed for sites in each cluster.

After fitting model parameters using stochastic variational inference, we re-added all sites that were filtered from the model above to the data set and solved for the optimal cluster assignment parameters for every autosomal site by fully enumerating all latent variables, using Pyro's enumeration strategy for discrete latent variables, with a guide function that fixed the learned model parameters but allowed assignment probabilities to vary. We then estimated the likelihood of each possible fetal genotype by summing the cluster component assignment probabilities: the likelihood that the fetal genotype is 0/0 (ref/ref) at the site was the probability of the site's assignment to cluster 1; the likelihood of a 0/1 (ref/alt) fetal genotype is the sum of the assignment probabilities for clusters 0, 2, and 4; and the likelihood of a 1/1 (alt/alt) fetal genotype is the assignment probability for cluster 3. Sites that appeared to be homozygous alternate in the cfDNA sample (i.e., for which the VAF was greater than 0.975) were automatically assigned a homozygous alternate genotype. Similarly, maternal genotype likelihoods were set as follows: the likelihood of a maternal 0/0 genotype was set to the assignment probability for cluster 0; the likelihood of a maternal 0/1 genotype was set to the sum of the assignment probabilities for clusters 1, 2, and 3; and the likelihood of a maternal 1/1 genotype was set to the assignment probability for cluster 4.

We applied this model to all autosomal variants in every sample, and to variants on chromosome X in samples in which the fetal sex chromosome ploidy was predicted to be XX. For samples with predicted fetal sex chromosome ploidy of XY, we used the model to genotype variants only within the pseudoautosomal regions (PAR) of chromosome X. For chromosome X variants outside of the PAR in XY samples, we defined three gaussian components for each possible pair of maternal and fetal genotypes (excluding variants homozygous in both mother and fetus). We defined these components based on the parameters learned in training the autosomal model as follows: for the cluster representing maternal heterozygous variants where the fetus carries the variant, the VAF mean was set to 1/(2−f); for the cluster representing maternal heterozygous variants where the fetus does not carry the variant, the VAF mean was set to (1−f)/(2−f); and a third cluster represents variants that are homozygous reference and variant in the fetus (i.e. de novo mutations) with VAF mean f/(2−f). The fragment size means for these clusters were set to the means learned in the autosomal model for clusters 1, 3, and 0, respectively, with a variance equal to the fragment size variance from autosomal cluster 0 times 5 (to account for additional variation observed at these sites). We assigned genotypes to these variants by computing the likelihood that each variant was generated by each of these Gaussian components and assigning the variant to that cluster's genotype set accordingly.

After genotyping, we applied two more filters to the resulting variant calls. First, we filtered out calls where the variant allele fraction was too low to have been generated by the cluster representing variants where the fetus is heterozygous and the mother is homozygous reference, cluster 0. To do this, we conducted a lower-tailed binomial distribution test of the observed number of reads supporting the alternate allele out of the total depth at the site, with a binomial probability of f/2, the expected VAF for that cluster, and filtered out any sites where the p-value of this test was less than 1e-5. Second, we filtered out any indel calls where the alternate allele was supported by three or fewer reads, as we found a high error rate in these variants.

Truth Data Processing: Standard ES from gDNA Variant Calling in Maternal, Paternal, Fetal Cord Blood, and Amniocentesis Samples

The gDNA libraries were prepared from maternal, paternal, fetal cord blood, and amniocentesis samples following standard ES protocols at the Broad Institute Genomics Platform (Cambridge, MA). After Illumina sequencing, reads were aligned, and variants were called following GATK best practices guidelines²⁵. Briefly, following marking and clipping of adapter sequences, pre-processed reads were aligned to the human reference using BWA-MEM²⁴ with default parameters. Duplicate reads were marked using Picard MarkDuplicates and excluded from downstream analysis. Base recalibration was performed using GATK BaseRecalibrator and ApplyBQSR (using known sites of variation from the GATK Reference Bundle). Germline single-nucleotide variants (SNVs) and indels were called for each sample using GATK HaplotypeCaller in GVCF mode followed by joint genotyping across all maternal and fetal DNA derived samples and variant filtration with GATK VQSR. To ensure a high-quality set of genotypes for use in benchmarking, we further applied a stringent set of variant filters previously used in large scale familial sequencing projects³². Briefly, variant sites were removed if they overlapped low complexity regions of the genome; variant genotypes were filtered that met any of the following criteria: depth less than 10; allele balance <0.25 or >0.75; probability of the allele balance (based on a binomial distribution with mean 0.5) below 1e-9; or fewer than 90 of the reads being informative for genotype. For the amniocentesis sample for study participant MGB043, sequencing was performed at Boston Children's Hospital (Boston, MA) using protocols from GeneDx (Stamford, CT). Sequencing data from this sample was re-aligned to hg38 and then re-processed according to the informatics steps listed above; for this sample alone, we limited benchmarking evaluations to the intersection of the exome target regions of the Broad Custom Exome kit used for the rest of the samples and the GeneDx kit.

### Benchmarking and Evaluations

Variants were compared to “truth” genotype data derived from ES of gDNA from either matched cord blood, amniocentesis, maternal DNA collected from leukocytes, or paternal samples (see section gDNA ES Variant Calling in Maternal, Paternal, Fetal Cord, and Amniocentesis Samples). For the comparison of cfDNA variants to cord blood or amniocentesis, we conducted five sets of evaluations, reported in Tables 5, 6, 8, and 10:
A site-level comparison of variants that were not removed by our filtering method (see section “cfDNA Variant Filtering”) that did not consider the fetal genotype at the site (Table 10, “After Filter Variant Detection”). This evaluation provides an assessment of the limits to sensitivity of cfDNA sequencing at the depths used in this study, after an attempt to remove sequencing artifacts and other errors from the sequencing data. As with the Unfiltered Variant Detection evaluation below, we excluded maternal variants that were not transmitted to the fetus from this evaluation so that the PPV metrics show the ability of the method to distinguish errors from true biological variation.
A site-level comparison of all variant sites detected in the cfDNA sequencing data (and therefore of either maternal or fetal origin, or both) without regard to the ultimate filter status or fetal genotype assigned to the site by our bioinformatic pipelines (also in Table 10, “Unfiltered Variant Detection”). This evaluation provides an assessment of the theoretical limits to sensitivity of cfDNA sequencing at the depths used in this study without attempting to remove sequencing artifacts and other errors. We excluded any sites which were present in the mother but not transmitted to the fetus (according to the maternal and cord blood or amniocentesis gDNA ES data) and therefore FPs in this evaluation are expected to represent true sequencing or mapping errors, as opposed to failures in fetal/maternal genotyping.
A comparison of all fetal genotypes assigned by our model to all genotypes called in the cord blood or amniocentesis ES data (Table 5, “Overall Genotyping Performance”). This evaluation assesses the accuracy of our genotyping model, which attempts to assign a fetal and maternal genotype to all sites detected in the cfDNA (which is a mixture of cfDNA fragments with maternal and fetal origins). See Supp. Methods section cfDNA Genotyping for a description of the genotyping model. In contrast to the “Unfiltered Variation Detection” and “After Filter Variant Detection” evaluations, untransmitted maternal variants are included. The results of this assessment represent the full ability of our informatic methods to determine the fetal genotype at every site in the exome given only a cfDNA sequencing sample.
A comparison of all variants that were assigned a fetal heterozygous genotype and a maternal homozygous reference genotype (Table 6, “Predicted Paternal or de novo Variant Detection”) to sites that were present in the cord blood or amniocentesis gDNA ES data but were not present in the maternal gDNA ES data for that participant. In this evaluation, we excluded any variant sites detected in the maternal gDNA ES data from evaluation, and only assessed variants called in the NIFS data which the genotyping pipeline had assigned to the “Fetal 0/1; Maternal 0/0” cluster. This evaluation characterizes the method's accuracy in detecting paternally inherited variants, as well as de novo mutations.
An assessment of the ability of our methods to accurately genotype variants that are heterozygous in the mother (Table 8, “NIFS Genotype Accuracy for Variants Heterozygous in the Mother”). This evaluation focuses only on sites where the maternal gDNA ES data indicates that the mother is heterozygous for a variant with passing filter status. These sites are important for recessive disease diagnostics but are more difficult to genotype a low fetal fraction. For this evaluation we report a single accuracy metric which is the percentage of true maternal heterozygous sites that were assigned a passing filter status and the correct fetal genotype by NIFS.

All the above evaluations except for “NIFS Genotype Accuracy for Variants Heterozygous in the Mother” were conducted with the vcfeval tool from Real Time Genomics^{33,34} (RTG; realtimegenomics.com/products/rtg-tools), which conducts a haplotype-based analysis to match variants between samples, and is a widely accepted standard for genomic variant calling evaluations. All benchmarking analyses were limited to intervals targeted by the exome capture panel on the autosomes. The “Unfiltered Variant Detection” and “After Filtering Variant Detection” evaluations in the comparison to cord blood and amniocentesis samples were conducted by matching sites without respect to the called genotype. In these two evaluations the presence of the same variant site, matched on genomic position and alternate allele, in both the cfDNA sample and the confirmation data counted as a true positive (this was achieved using the vcfeval parameter—squash-ploidy). The “Overall Genotyping Performance” comparison, on the other hand, required each called fetal allele in the output of our pipeline to match the alleles present in the genotypes called in the confirmation data. Benchmarking evaluates true positives (TP), FP, true negatives (TN), and false negatives (FN). Sensitivity and PPV were calculated by RTG vcfeval as:

$$PPV = TP / ( TP + FP )$$

$$Sensitivity = TP / ( TP + FN )$$

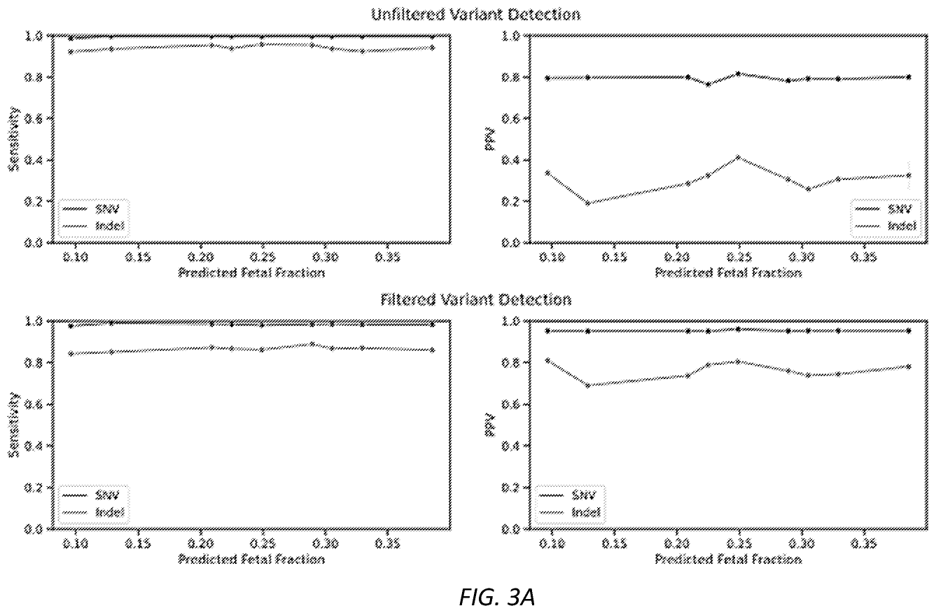

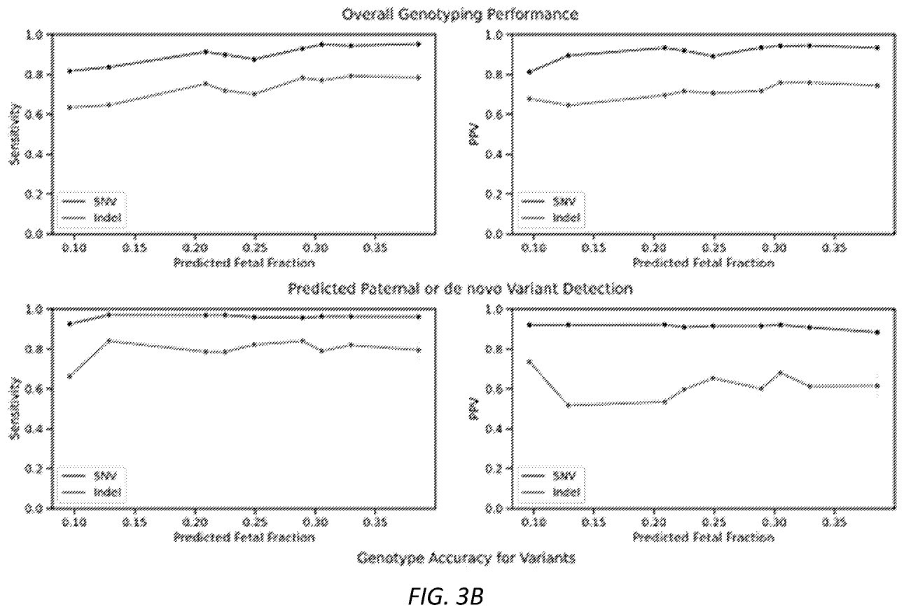

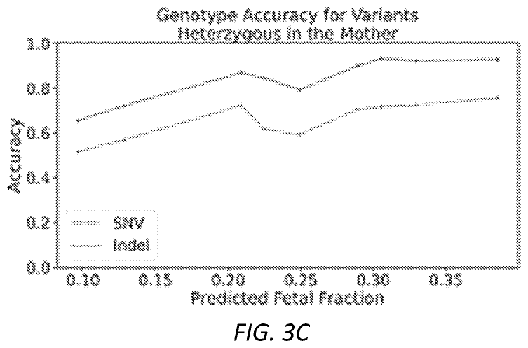

For all of the above evaluations except for “Genotype Accuracy for Variants Heterozygous in the Mother”, we excluded from evaluation all regions where the ES data from the cord blood or amniocentesis samples had coverage of less than 10 reads—in other words, called variant sites in these regions were not counted as TP, FP, or FN. This evaluation was conducted using the same analysis scripts as were used for the “Genotype Accuracy by Maternal and Fetal Genotype” reported in Table 9, described below. We note that the metrics presented in this evaluation can be computed by summarizing the results for each of the genotype clusters corresponding to maternal heterozygous variants in Table 9. See also FIGS. 3A-C.

In addition to the analyses described above, we also used the matched cord blood or amniocentesis gDNA ES data for a more detailed breakdown of NIFS' sensitivity and genotype accuracy on all confirmed variants in the fetal and maternal exomes (Table 9, “Genotype Accuracy by Maternal and Fetal Genotype”). For this evaluation, we compared all NIFS calls made from cfDNA to the union set of all variants called in either the maternal gDNA ES or the cord blood/amniocentesis gDNA ES. For each combination of maternal and fetal genotype present in this comparison set of maternal and fetal variants, we calculated the percentage of sites with matching positions and alternate allele that were present in the raw Mutect2 cfDNA VCF (as reported in Table 10) and the percentage of those sites that were not filtered and were assigned the correct fetal genotype by the cfDNA variant calling pipeline. These evaluations were conducted with a custom analysis script which matched variant calls in the maternal gDNA ES, cord blood or amniocentesis gDNA ES, and cfDNA sequencing data by genomic position and alternate allele (as opposed to the haplotype-based methods implemented in RTG vcfeval).

A second set of evaluations compared the maternal genotypes predicted by our model to the variants detected in ES sequencing of maternal gDNA extracted from precipitated maternal leukocytes. The results of this evaluation are reported in Table 11 in two parts, “Detection of Maternal Variants” and “Maternal Genotyping Performance”. We allowed any called variant sites to match for the “Detection of Maternal Variants” comparison, regardless of the maternal genotypes assigned by NIFS or gDNA variant calling. We required full genotype matches between gDNA and ES calls for the “Maternal Genotyping Performance” evaluation. For these maternal evaluations we excluded any sites for which the maternal gDNA ES data had less than 1Ox read coverage. These evaluations were conducted using the RTG vcfeval tool.

Finally, for participants with matching gDNA ES data derived from a paternal blood sample, we conducted an evaluation of the proportion of sites assigned a non-reference fetal genotype in the cfDNA data, excluding sites that were present in the maternal gDNA ES data, which were present in the paternal gDNA ES data. The results of this evaluation are reported in Table 7, “Genotyping Performance and Coverage Based on Paternal gDNA ES”. This evaluation is another way of computing the PPV of NIFS calls that are predicted to be either paternally inherited or de novo mutations in addition to the “Predicted Paternal or De Novo Variant Detection” results reported in Table 6. For this analysis, we used RTG vcfeval to calculate the PPV of all calls assigned to cluster 0 (the cluster representing fetal heterozygous and maternal homozygous reference variants) against the set of paternal ES variants, and we limited the evaluation to sites that did not match a variant site in the maternal ES data. We excluded any regions where the paternal ES data had read coverage of less than 10× from this evaluation. We also report the number of reads supporting the alternate allele for each of these confirmed paternal variants detected by NIFS.

For sample MGB043, the amniocentesis sample was sequenced at Boston Children's Hospital using a different exome capture kit provided by GeneDx, and we therefore limited all evaluations to the set of exome target intervals covered by both the Twist Custom Exome list used for the NIFS samples and the GeneDx exome targets (n=194,202 intervals).

### Familial Relationship Inference

Predicted genetic relationships (between cfDNA, parental, and cord blood and amniocentesis samples) were confirmed with KING³⁵ after variant calling. To confirm suspected familial relationships in our cohort we filtered the cfDNA variants to include only those with a gnomAD allele frequency (AF_popmax) greater than 0.05 and a quality score for fetal genotype inference greater than 10. Processing the resulting predicted genotyping cluster with KING (parameters-related-degree 2), verified that the expected relationships had an estimated proportion of the genome identical by descent (KING metric PropIBD) of at least 0.4 (with one exception, a paternal-fetal pair with 0.32 propIBD, which we manually confirmed).

### Detection of Copy Number Variants (CNVs)

We developed a sliding-window binning approach to investigate significant deviations in copy state using coverage collected from GATK CollectReadCounts with GC correction. Copy states were normalized against a subset of the control NIFSs libraries (absent fetal anomaly cases, Table 12) with GATK CreateReadCountPanelOfNormals and DenoiseReadCounts. We filtered out highly variable capture intervals with median absolute deviations (MAD) greater than 3rd quartile+1.5*interquartile range (IQR) in the control cfDNA samples. We then computed the median copy ratio for each sample in bins representing sliding windows across the genome of size 3 MB with an offset of 100 kb. A final filtering step was applied, removing 1 MB bins with >10 of control samples classified as outliers based on a per-bin IQR analysis. Only one validated CNV event was observed in our study cohort, so we were unable to conduct extensive benchmarking or a sensitivity analysis of CNVs. We note that our previous gDNA ES studies, as reported by Fu et al.³², have demonstrated accurate CNV discovery beyond the resolution of individual genes—down to routine discovery of events that span >2 exons—and have noted the potential for discovery of CNVs at single exon resolution. Detection of these events in cfDNA will be difficult due to the mixture of maternal and fetal DNA, but more data will allow for the development of improved methods and thorough benchmarking.

### Sex Determination

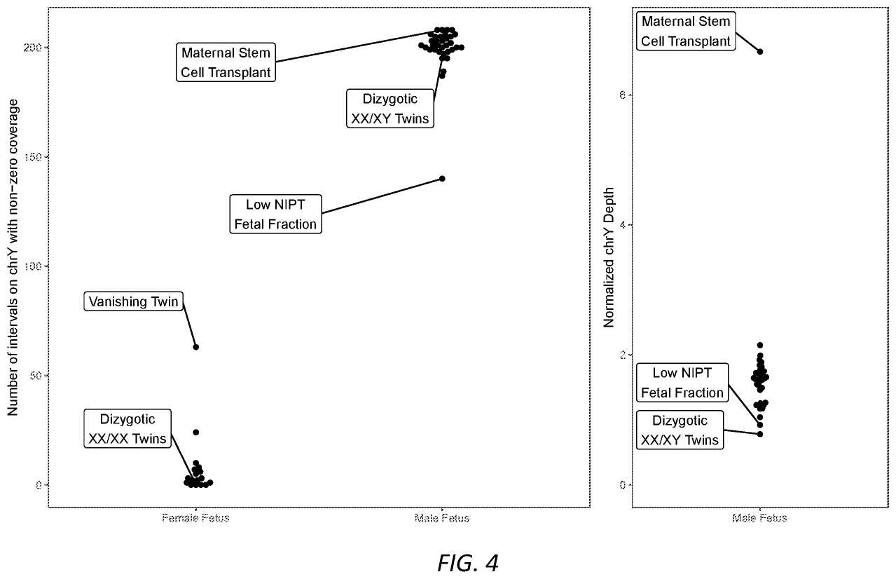

We explored the ability of NIFS to determine fetal sex given the robust coverage of chrY and chrX. We initially focused on chrY for delineation of sex given that any reads on chrY, beyond a few artifacts, should indicate male fetal sex. In fact, the presence of any coverage (from GATK CollectReadCounts), on chrY binned interval was highly discriminatory for sex determination (FIG. 4), though exact prediction of chrY copy state determined by dividing the median coverage across all intervals chrY by fetal fraction, remained challenging due to the relatively low and variable coverage on chrY compared with the rest of the genome.

### Variant Classification

We analyzed each sample for potentially pathogenic variation in the fetus and mother, using genotypes derived from the cfDNA results. We applied bcftools³⁶ merge to create a multisample VCF of all samples with cffDNA sequencing. Using ANNOVAR³⁷ and bcftools, this merged VCF was annotated with genic and functional consequences (RefSeq²⁶), allele frequency (gnomAD v2.1.1 and gnomAD v3.0), REVEL³⁸ scores, ClinVar³⁹ annotations (updated 2023 Apr. 30), and per gene disease information such as inheritance type (e.g. recessive) from the Online Mendelian Inheritance in Man (OMIM, version 2022-07-08⁴⁰). We included variants if they had an allele frequency of <5 or were not reported in gnomAD v2.1.1 and gnomAD v3.0²⁸, and excluded synonymous variants. We then created a list from each sample for further review, including all ClinVar annotated Pathogenic/Likely Pathogenic variants, all frameshift/stopgain variants, all predicted splice variants with a Splice Al score⁴¹>0.95, all non-frameshift variants >15 amino acids; and all non-synonymous variants with a REVEL score >0.7. With the exception of ClinVar P/LP variants, variants not passing filters were removed. Of this set, variants with <4 alternate reads, and those determined likely_benign or benign/likely_benign in ClinVar were filtered.

We ascertained fetal genotype using the methods described above with the caveat that for a small subset of indels that were phased with a high quality SNV, we use the SNV genotype given the higher SNV genotype accuracy. Variants in disease genes from OMIM were selected for further analysis. We manually reviewed each of the remaining variants using the Integrated Genomics Viewer (IGV) and removed variants that appeared to be low quality or were present in multiple NIFS samples (indicating that they were likely technical artifacts). Variants were reviewed for pathogenicity based on ACMG criteria⁴²⁻⁴⁴ and clinical relevance was assessed. CNVs were assessed following Clingen and ACMG guidelines provided by Riggs et al.⁴⁵. We assessed potential carrier variants for the 28 samples with matching maternal germline exome sequencing data (Table 3), we further filtered these based on their ClinVar pathogenicity. Variants were considered if they were listed as pathogenic or likely pathogenic in ClinVar with Clinical Significance corresponding to 2 or more gold stars (i.e. practice guideline, reviewed by expert panel, or criteria provided with multiple submitters and no conflicts). Variants with genotypes corresponding to maternal carrier status were selected. As before, variants were reviewed for potential clinical relevance (Table 14). All identified variants were confirmed by maternal germline ES.

TABLE 1
Characteristics of Study Samples

| Study | | | |
|---|---|---|---|
| Characteristics: | | | |
| Type of Pregnancy | N | XY | XX |
| Singleton | 49 | 33 | 16 |
| Twin: Monozygotic | 1 pair | 0 | 2 |
| Twin: Dizygotic | 1 pair | 1 | 1 |

| Fetal Fraction: | Median | IQR | Min-Max |
|---|---|---|---|
| Percentage of fetal | 25 | 14-30 | 6-51 |
| cffDNA | | | |

| Gestational Age: | 1st | 2nd | 3rd |
|---|---|---|---|
| Trimester | 5 | 9 | 37 |

| Benchmarking/ | Parental Germline DNA | Cord | |
|---|---|---|---|
| Confirmation Data: | (Maternal/Paternal) | Blood | Amniocentesis |
| Germline ES Count | 28/7 | 7 | 4 |

| NIFS Library | | | |
|---|---|---|---|
| Characteristics: | Mean | IQR | Min-Max |
| Average Per Sample | 210 | 165-232 | 101-467 |
| Mean Sequencing | | | |
| Coverage | | | |
| Duplication Rate | 59 | 52-63 | 40-83 |

IQR—interquartile range

TABLE 2
Representativeness of Study Participants

| CATEGORY | RESPONSE |
|---|---|
| DISEASE, PROBLEM, OR | cfDNA testing for fetal genetic diseases |
| CONDITION UNDER | on maternal plasma in pregnancy. |
| INVESTIGATION: | |
| SPECIAL CONSIDERATIONS | |
| RELATED TO: | |
| SEX AND GENDER | Pregnant individuals who all identified |
| | as women. |
| AGE | Reproductive age of women. |
| RACE OR ETHNIC | Race and ethnicity of this pregnant |
| GROUP | population is representative of the |
| | pregnant population at the recruitment |
| | hospital. |
| GEOGRAPHY | US-based population receiving prenatal |
| | care in the Boston area. |
| OTHER | The participants had a higher education |
| CONSIDERATIONS | level and older maternal age than the |
| | overall US pregnant population. |
| OVERALL | Participants were representative of the |
| REPRESENTATIVENESS | pregnant population receiving care at |
| OF THIS TRIAL | the hospital of recruitment. |

TABLE 3
Sample Information

| | Trimester | Estimated | | Maternal | Paternal | |
|---|---|---|---|---|---|---|
| Sample | of | Fetal | Fetal | Germline | Germline | Confirmation |
| ID | Sample | Fraction | Sex | ES | ES | Sample ES |
| MGB1 | 3rd | 0.26 | XY | No | No | No |
| MGB2 | 3rd | 0.30 | XX | No | No | No |
| MGB3 | 3rd | 0.27 | XY | No | No | No |
| MGB4 | 3rd | 0.26 | XY | No | No | No |
| MGB5 | 3rd | 0.14 | XY | No | No | No |
| MGB6 | 3rd | 0.25 | XY | No | No | No |
| MGB7 | 3rd | 0.22 | XY | No | No | No |
| MGB8 | 3rd | 0.12 | XX | No | No | No |
| MGB9 | 3rd | 0.18 | XY | No | No | No |
| MGB10 | 3rd | 0.25 | XY | No | No | No |
| MGB11 | 3rd | 0.13 | XY | No | No | No |
| MGB12 | 3rd | 0.30 | XX | No | No | No |
| MGB13 | 3rd | 0.14 | XX | No | No | No |
| MGB14 | 3rd | 0.30 | XX | No | No | No |
| MGB15 | 3rd | 0.20 | XX/XY | No | No | No |
| MGB16 | 3rd | 0.50 | XX | No | No | No |
| MGB17 | 3rd | 0.50 | XX | No | No | No |
| MGB18 | 3rd | 0.25 | XY | No | No | No |
| MGB19 | 3rd | 0.40 | XX | No | No | No |
| MGB20 | 3rd | 0.39 | XY | Yes | No | Cord Blood |
| MGB21 | 3rd | 0.20 | XY | No | No | No |
| MGB22 | 3rd | 0.40 | XX | Yes | No | Cord Blood |
| MGB23 | 3rd | 0.28 | XY | No | No | No |
| MGB24 | 3rd | 0.30 | XY | No | No | No |
| MGB25 | 3rd | 0.36 | XY | No | No | No |
| MGB26 | 3rd | 0.25 | XY | Yes | No | Cord Blood |
| MGB27 | 3rd | 0.24 | XX | Yes | No | Cord Blood |
| MGB28 | 3rd | 0.30 | XX | Yes | No | No |
| MGB29 | 3rd | 0.30 | XY | Yes | No | Cord Blood |
| MGB30 | 3rd | 0.34 | XY | Yes | No | No |
| MGB31 | 3rd | 0.32 | XX | Yes | No | Cord Blood |
| MGB32 | 3rd | 0.28 | XX | Yes | No | No |
| MGB33 | 3rd | 0.30 | XY | Yes | No | No |
| MGB34 | 2nd | 0.13 | XY | Yes | No | No |
| MGB35 | 2nd | 0.14 | XX | Yes | No | No |
| MGB36 | 1st | 0.14 | XY | Yes | No | No |
| MGB37 | 1st | 0.08 | XY | Yes | No | No |
| MGB38 | 2nd | 0.22 | XY | Yes | Yes | Amniocentesis |
| MGB39 | 1st | 0.08 | XY | Yes | Yes | No |
| MGB40 | 2nd | 0.20 | XX | Yes | Yes | No |
| MGB41 | 2nd | 0.09 | XY | Yes | Yes | Amniocentesis |
| MGB42 | 3rd | 0.30 | XY | Yes | No | Amniocentesis |
| MGB43 | 2nd | 0.12 | XX/XX | Yes | Yes | Amniocentesis |
| MGB44 | 3rd | 0.51 | XY | Yes | No | No |
| MGB45 | 3rd | 0.29 | XY | Yes | No | Cord Blood |
| MGB46 | 1st | 0.1 | XY | Yes | No | No |
| MGB47 | 3rd | 0.19 | XY | Yes | No | No |
| MGB48 | 2nd | 0.06 | XY | Yes | No | No |
| MGB49 | 2nd | 0.09 | XX | Yes | No | No |
| MGB50 | 2nd | 0.16 | XY | Yes | Yes | No |
| MGB51 | 1st | 0.14 | XY | Yes | Yes | No |

TABLE 4
Sample Coverage and Sequencing Metrics

| | | | % of | | |
|---|---|---|---|---|---|
| | | | Target | % of Targets | % of Targets |
| | | | Bases | with | with |
| | | | with at | Estimated | Estimated |
| | Mean | Estimated | Least | 8X Mean | 10X Mean |
| Sample | Cover- | Duplication | 50X Total | Fetal Read | Fetal Read |
| ID | age | Rate | Coverage | Coverage | Coverage |
| MGB1 | 135.2 | 63% | 96.32% | 97.95% | 97.56% |
| MGB2 | 110.9 | 61% | 93.87% | 97.71% | 97.13% |
| MGB3 | 143.8 | 61% | 96.39% | 97.98% | 97.65% |
| MGB4 | 101.6 | 60% | 92.20% | 97.04% | 95.57% |
| MGB5 | 119.3 | 62% | 95.06% | 92.90% | 85.97% |
| MGB6 | 168.0 | 57% | 95.59% | 97.78% | 97.16% |
| MGB7 | 174.6 | 50% | 93.30% | 95.92% | 93.32% |
| MGB8 | 173.2 | 49% | 94.19% | 86.16% | 77.51% |
| MGB9 | 202.1 | 47% | 92.47% | 92.63% | 88.37% |
| MGB10 | 192.3 | 49% | 94.52% | 97.19% | 95.96% |
| MGB11 | 159.5 | 65% | 96.27% | 93.93% | 89.08% |
| MGB12 | 221.6 | 52% | 96.61% | 98.04% | 97.83% |
| MGB13 | 215.6 | 58% | 96.64% | 95.83% | 93.03% |
| MGB14 | 139.8 | 53% | 94.33% | 97.58% | 96.89% |
| MGB15 | 307.8 | 50% | 97.29% | 97.89% | 97.45% |
| MGB16 | 214.3 | 51% | 97.04% | 98.25% | 98.21% |
| MGB17 | 228.3 | 56% | 97.14% | 98.25% | 98.21% |
| MGB18 | 202.2 | 60% | 96.97% | 98.01% | 97.73% |
| MGB19 | 184.2 | 57% | 97.00% | 98.17% | 98.11% |
| MGB20 | 160.8 | 76% | 96.81% | 98.16% | 98.07% |
| MGB21 | 344.1 | 46% | 97.03% | 97.75% | 97.10% |
| MGB22 | 268.3 | 52% | 97.15% | 98.19% | 98.13% |
| MGB23 | 128.4 | 83% | 96.16% | 97.99% | 97.79% |
| MGB24 | 173.5 | 79% | 97.39% | 98.19% | 98.10% |
| MGB25 | 465.4 | 62% | 97.48% | 98.18% | 98.09% |
| MGB26 | 104.6 | 81% | 93.84% | 96.87% | 95.85% |
| MGB27 | 209.9 | 62% | 96.59% | 97.74% | 97.37% |
| MGB28 | 265.7 | 58% | 97.24% | 98.01% | 97.88% |
| MGB29 | 320.7 | 55% | 96.64% | 96.99% | 96.71% |
| MGB30 | 188.5 | 56% | 94.83% | 97.65% | 97.33% |
| MGB31 | 229.6 | 57% | 96.73% | 97.55% | 97.33% |
| MGB32 | 229.8 | 62% | 96.87% | 97.86% | 97.66% |
| MGB33 | 198.8 | 45% | 96.84% | 98.18% | 98.01% |
| MGB34 | 157.9 | 57% | 96.59% | 95.59% | 92.56% |
| MGB35 | 167.7 | 54% | 95.89% | 95.36% | 92.75% |
| MGB36 | 131.3 | 74% | 95.89% | 95.64% | 93.56% |
| MGB37 | 204.0 | 50% | 97.08% | 89.00% | 79.02% |
| MGB38 | 277.2 | 63% | 97.36% | 97.95% | 97.76% |
| MGB39 | 220.5 | 65% | 96.30% | 91.60% | 86.64% |
| MGB40 | 202.2 | 72% | 93.81% | 92.91% | 91.91% |
| MGB41 | 215.6 | 70% | 94.14% | 87.59% | 83.80% |
| MGB42 | 210.0 | 51% | 97.22% | 98.20% | 98.08% |
| MGB43 | 330.9 | 55% | 97.62% | 97.75% | 97.38% |
| MGB44 | 102.4 | 75% | 94.42% | 98.16% | 98.06% |
| MGB45 | 201.7 | 61% | 96.55% | 97.66% | 97.36% |
| MGB46 | 293.1 | 61% | 97.32% | 96.58% | 95.62% |
| MGB47 | 247 | 67% | 97.18% | 97.90% | 97.54% |
| MGB48 | 319.6 | 40% | 93.69% | 72.38% | 64.37% |
| MGB49 | 214.5 | 56% | 96.40% | 91.73% | 87.08% |
| MGB50 | 248.5 | 50% | 97.24% | 97.47% | 96.60% |
| MGB51 | 260.1 | 61% | 92.53% | 89.38% | 87.83% |

TABLE 5
Overall Genotyping Performance

| | | | | Overall NIFS Genotyping Performance (%)* | | | | | | | | | |
|---|---|---|---|---|---|---|---|---|---|---|---|---|---|
| | Mean | Fetal | | Total Variants | | | | | | | | | |
| | Target | Frac. | Confirmation | SNV | | | Indel | | | SNV | | Indel | |
| Sample | Cov. | (%) | Method | TP | FP | FN | TP | FP | FN | Sens. | PPV | Sens. | PPV |
| MGB22 | 269X | 40 | Cord ES | 20,887 | 1,446 | 1,073 | 324 | 122 | 96 | 95.1 | 93.5 | 77.3 | 72.7 |
| MGB20 | 161X | 39 | Cord ES | 20,911 | 1,501 | 968 | 354 | 110 | 90 | 95.6 | 93.3 | 79.8 | 76.3 |
| MGB31 | 232X | 32 | Cord ES | 20,980 | 1,213 | 1,219 | 320 | 101 | 85 | 94.5 | 94.5 | 79.2 | 76.0 |
| MGB29 | 323X | 30 | Cord ES | 20,654 | 1,230 | 1,063 | 331 | 105 | 99 | 95.1 | 94.4 | 77.1 | 75.9 |
| MGB42 | 211X | 30 | Amniocentesis | 20,072 | 1,417 | 1,521 | 336 | 131 | 88 | 93.0 | 93.4 | 79.3 | 72.0 |
| | | | ES | | | | | | | | | | |
| MGB45 | 203X | 29 | Cord ES | 20,878 | 1,389 | 1,559 | 331 | 131 | 98 | 93.1 | 93.8 | 77.3 | 71.7 |
| MGB26 | 104X | 25 | Cord ES | 18,896 | 2,286 | 2,662 | 298 | 124 | 128 | 87.6 | 89.2 | 70.2 | 70.6 |
| MGB27 | 195X | 24 | Cord ES | 19,040 | 1,660 | 2,123 | 305 | 121 | 120 | 90.0 | 92.0 | 71.9 | 71.6 |
| MGB38 | 279X | 22 | Amniocentesis | 19,289 | 1,366 | 1,827 | 295 | 129 | 97 | 91.4 | 93.4 | 75.3 | 69.6 |
| | | | ES | | | | | | | | | | |
| MGB43 | 334X | 12 | Amniocentesis | 16,451 | 1,936 | 3,205 | 210 | 115 | 116 | 83.7 | 89.5 | 64.6 | 64.6 |
| | | | ES | | | | | | | | | | |
| MGB41 | 217X | 9 | Amniocentesis | 17,670 | 4,083 | 3,945 | 266 | 126 | 155 | 81.8 | 81.2 | 63.4 | 67.9 |
| | | | ES | | | | | | | | | | |
| | | | Mean: | 19,612 | 1,775 | 1,924 | 306 | 120 | 107 | 91.0 | 91.7 | 74.1 | 71.7 |
| | | | Median: | 20,072 | 1,446 | 1,559 | 320 | 122 | 98 | 93.0 | 93.4 | 77.1 | 71.7 |
| | | | Sum: | 215,728 | 19,527 | 21,165 | 3,370 | 1,315 | 1,172 | | | | |

*Evaluates the fetal genotypes assigned to each site by NIFS as compared to the confirmation sample's genotype.
All sites in the exome target regions with sufficient depth in the confirmation sample are considered.

TABLE 6
Predicted Paternal or de novo Variant Detection

| | | | | NIFS Predicted Paternal or de novo Variant Detection (%)* | | | | | | | | | |
|---|---|---|---|---|---|---|---|---|---|---|---|---|---|
| | Mean | Fetal | | Total Variants | | | | | | | | | |
| | Target | Fraction | Confirmation | SNV | | | Indel | | | SNV | | Indel | |
| Sample | Coverage | (%) | Method | TP | FP | FN | TP | FP | FN | Sens. | PPV | Sens. | PPV |
| MGB22 | 269X | 40 | Cord ES | 4,264 | 585 | 185 | 71 | 58 | 24 | 95.8 | 87.9 | 74.7 | 55.0 |
| MGB20 | 161X | 39 | Cord ES | 4,515 | 568 | 162 | 91 | 43 | 17 | 96.5 | 88.8 | 84.3 | 67.9 |
| MGB31 | 232X | 32 | Cord ES | 4,409 | 445 | 171 | 76 | 48 | 17 | 96.3 | 90.8 | 81.9 | 61.3 |
| MGB29 | 323X | 30 | Cord ES | 4,411 | 381 | 168 | 83 | 39 | 22 | 96.3 | 92.1 | 79.1 | 68.0 |
| MGB42 | 211X | 30 | Amnio-centesis | 4,615 | 422 | 196 | 85 | 66 | 17 | 95.9 | 91.6 | 83.3 | 56.3 |
| | | | ES | | | | | | | | | | |
| MGB45 | 203X | 29 | Cord ES | 4,684 | 432 | 227 | 99 | 56 | 18 | 95.4 | 91.6 | 84.6 | 63.9 |
| MGB26 | 104X | 25 | Cord ES | 4,385 | 406 | 182 | 83 | 44 | 18 | 96.0 | 91.5 | 82.2 | 65.4 |
| MGB27 | 195X | 24 | Cord ES | 4,289 | 426 | 132 | 77 | 52 | 21 | 97.0 | 91.0 | 78.6 | 59.7 |
| MGB38 | 279X | 22 | Amnio-centesis | 4,306 | 364 | 134 | 77 | 67 | 21 | 97.0 | 92.2 | 78.6 | 53.5 |
| | | | ES | | | | | | | | | | |
| MGB43 | 334X | 12 | Amnio-centesis | 3,905 | 335 | 118 | 62 | 58 | 12 | 97.1 | 92.1 | 84.0 | 51.7 |
| | | | ES | | | | | | | | | | |
| MGB41 | 217X | 9 | Amnio-centesis | 4,269 | 368 | 343 | 75 | 27 | 38 | 92.6 | 92.1 | 66.4 | 73.5 |
| | | | ES | | | | | | | | | | |
| | | | Mean: | 4,368 | 430 | 183 | 80 | 51 | 20 | 96.0 | 91.1 | 79.8 | 61.5 |
| | | | Median: | 4,385 | 422 | 171 | 77 | 52 | 18 | 96.3 | 91.6 | 81.9 | 61.3 |
| | | | Sum: | 48,052 | 4,732 | 2,018 | 879 | 558 | 225 | | | | |

*Evaluates all sites that NIFS predicts to be heterozygous in the fetus and not present in the mother against sites that are present in the confirmation sample and not present in the maternal gDNA sample.
Excludes regions with coverage of less than 10x in the confirmation sample.
Bold indicates values highlighted in letter.

TABLE 7
Genotyping Performance and Coverage Based on Paternal gDNA ES

| | | | | Non-maternal sites | | | | Number of NIFS reads | | | |
|---|---|---|---|---|---|---|---|---|---|---|---|
| | | | | called by NIFS that were | | | | supporting paternal allele at | | | |
| | | | | present in paternal | | | | non-maternal sites called by | | | |
| | Mean | Fetal | | gDNA ES data | | | | NIFS | | | |
| | Target | Fraction | Confirmation | SNV | | Indel | | SNV | | Indel | |
| Sample | Coverage | (%) | Method | # | % | # | % | Median | IQR | Median | IQR |
| MGB38 | 279X | 22 | Paternal ES & | 4,305 | 95.6 | 73 | 64.6 | 27 | [20-35] | 22 | [13-31] |
| | | | Maternal ES | | | | | | | | |
| MGB40 | 203X | 20 | Paternal ES & | 3,954 | 93.4 | 65 | 70.7 | 20 | [15-26] | 17 | [12-23] |
| | | | Maternal ES | | | | | | | | |
| MGB50 | 251X | 16 | Paternal ES & | 4,235 | 93.8 | 69 | 63.9 | 17 | [11-24] | 16 | [11-22] |
| | | | Maternal ES | | | | | | | | |
| MGB43 | 334X | 12 | Paternal ES & | 3,935 | 95.0 | 66 | 60.6 | 19 | [13-25] | 18 | [11-22] |
| | | | Maternal ES | | | | | | | | |
| MGB41 | 217X | 9 | Paternal ES & | 4,257 | 93.8 | 74 | 76.3 | 10 | [7-13] | 9 | [6-12] |
| | | | Maternal ES | | | | | | | | |
| MGB51 | 262X | 14 | Paternal ES & | 4,322 | 95.5 | 73 | 72.3 | 18 | [11-25] | 15 | [9-20] |
| | | | Maternal ES | | | | | | | | |
| MGB39 | 221X | 8 | Paternal ES & | 3,777 | 91.1 | 42 | 52.5 | 6 | [4-8] | 7 | [6-9] |
| | | | Maternal ES | | | | | | | | |
| | | | Mean: | 4,112 | 94.0 | 66.0 | 65.8 | 16.7 | | 14.9 | |
| | | | Median: | 4,235 | 93.8 | 69 | 64.6 | 18 | | 16 | |
| | | | Sum: | 28,785 | | 462 | | | | | |

TABLE 8
Genotyping Accuracy for Maternal Heterozygous Variants

| | | | | NIFS Genotyping Accuracy for | | | |
|---|---|---|---|---|---|---|---|
| | | | | Variants Heterozygous in the | | | |
| | Mean | Fetal | | Mother (%)* | | | |
| | Target | Fraction | Confirmation | Total Variants | | Accuracy | |
| Sample | Coverage | (%) | Method | SNV | Indel | SNV | Indel |
| MGB22 | 269X | 40 | Cord ES | 13,653 | 261 | 92.7% | 73.6% |
| MGB20 | 161X | 39 | Cord ES | 13,644 | 275 | 92.8% | 77.8% |
| MGB31 | 232X | 32 | Cord ES | 14,457 | 273 | 92.3% | 72.5% |
| MGB29 | 323X | 30 | Cord ES | 13,234 | 286 | 93.2% | 71.7% |
| MGB42 | 211X | 30 | Amniocentesis | 12,875 | 275 | 89.5% | 71.3% |
| | | | ES | | | | |
| MGB45 | 203X | 29 | Cord ES | 15,113 | 304 | 90.5% | 69.7% |
| MGB26 | 104X | 25 | Cord ES | 13,283 | 266 | 79.3% | 59.4% |
| MGB27 | 195X | 24 | Cord ES | 13,290 | 266 | 84.6% | 61.7% |
| MGB38 | 279X | 22 | Amniocentesis | 12,689 | 224 | 86.8% | 72.3% |
| | | | ES | | | | |
| MGB43 | 334X | 12 | Amniocentesis | 13,249 | 256 | 72.3% | 57.0% |
| | | | ES | | | | |
| MGB41 | 217X | 9 | Amniocentesis | 13,750 | 259 | 65.6% | 51.7% |
| | | | ES | | | | |
| | | | Mean: | 13,567 | 268 | 85.4% | 67.2% |
| | | | Median: | 13,290 | 266 | 89.5% | 71.3% |
| | | | Sum: | 149,237 | 2,945 | | |

*Evaluates the accuracy of the fetal genotypes assigned by NIFS for all sites which are heterozygous in the mother (as determined by the maternal gDNA ES).
Bold indicates values highlighted in letter.

TABLE 9
Genotyping Accuracy by Maternal and Fetal Genotype

| | | | | | | | | | SNV | | | Indel |
|---|---|---|---|---|---|---|---|---|---|---|---|---|
| | | | | | | Sites | | | Sites | | | Sites |
| | | | | | | Assigned | | | Assigned | | | Assigned |
| | | | | | | Correct | | SNV | Correct | | Indel | Correct |
| | Mean | Fetal | Fetal and | | Site- | Fetal | # | Site- | Fetal | # | Site- | Fetal |
| | Target | Frac. | Maternal | # | level | Genotype | SNV | level | Genotype | Indel | level | Genotype |
| Sample | Cov. | (%) | Genotype | Sites | Sens. | (%) | Sites | Sens. | (%) | Sites | Sens. | (%) |
| MGB22 | 269X | 40 | Fet. 0/1; | 4,558 | 99.5 | 95.4 | 4,457 | 99.7 | 95.9 | 101 | 91.1 | 72.3 |
| | | | Mat. 0/0 | | | | | | | | | |
| | | | Fet. 0/0; | 4,448 | 99.7 | 89.6 | 4,361 | 99.8 | 90.2 | 87 | 94.3 | 62.1 |
| | | | Mat. 0/1 | | | | | | | | | |
| | | | Fet. 0/1; | 7,200 | 99.9 | 94.3 | 7,069 | 99.9 | 94.6 | 131 | 97.7 | 80.9 |
| | | | Mat. 0/1 | | | | | | | | | |
| | | | Fet. 1/1; | 2,266 | 100.0 | 91.2 | 2,223 | 100.0 | 91.5 | 43 | 100.0 | 74.4 |
| | | | Mat. 0/1 | | | | | | | | | |
| | | | Fet. 0/1; | 2,122 | 99.7 | 90.7 | 2,081 | 99.8 | 91.3 | 41 | 95.1 | 61.0 |
| | | | Mat. 1/1 | | | | | | | | | |
| | | | Fet. 1/1; | 5,971 | 99.9 | 99.5 | 5,881 | 99.9 | 99.6 | 90 | 97.8 | 92.2 |
| | | | Mat. 1/1 | | | | | | | | | |
| MGB20 | 161X | 39 | Fet. 0/1; | 4,793 | 99.0 | 96.2 | 4,682 | 99.2 | 96.5 | 111 | 91.9 | 81.1 |
| | | | Mat. 0/0 | | | | | | | | | |
| | | | Fet. 0/0; | 4,839 | 99.3 | 90.8 | 4,738 | 99.4 | 91.1 | 101 | 94.1 | 76.2 |
| | | | Mat. 0/1 | | | | | | | | | |
| | | | Fet. 0/1; | 6,635 | 99.8 | 94.5 | 6,502 | 99.9 | 94.7 | 133 | 98.5 | 82.7 |
| | | | Mat. 0/1 | | | | | | | | | |
| | | | Fet. 1/1; | 2,445 | 99.9 | 90.4 | 2,404 | 99.9 | 90.9 | 41 | 97.6 | 65.9 |
| | | | Mat. 0/1 | | | | | | | | | |
| | | | Fet. 0/1; | 2,326 | 99.9 | 95.4 | 2,277 | 99.9 | 95.8 | 49 | 100.0 | 77.6 |
| | | | Mat. 1/1 | | | | | | | | | |
| | | | Fet. 1/1; | 5,916 | 99.8 | 99.2 | 5,824 | 99.8 | 99.4 | 92 | 97.8 | 89.1 |
| | | | Mat. 1/1 | | | | | | | | | |
| MGB31 | 232X | 32 | Fet. 0/1; | 4,685 | 99.1 | 95.9 | 4,586 | 99.4 | 96.3 | 99 | 86.9 | 76.8 |
| | | | Mat. 0/0 | | | | | | | | | |
| | | | Fet. 0/0; | 5,012 | 98.9 | 94.6 | 4,900 | 99.2 | 95.1 | 112 | 84.8 | 70.5 |
| | | | Mat. 0/1 | | | | | | | | | |
| | | | Fet. 0/1; | 7,483 | 99.8 | 89.5 | 7,361 | 99.8 | 89.7 | 122 | 95.9 | 73.0 |
| | | | Mat. 0/1 | | | | | | | | | |
| | | | Fet. 1/1; | 2,235 | 99.9 | 94.1 | 2,196 | 99.9 | 94.4 | 39 | 100.0 | 76.9 |
| | | | Mat. 0/1 | | | | | | | | | |
| | | | Fet. 0/1; | 2,383 | 99.8 | 97.7 | 2,347 | 99.9 | 97.8 | 36 | 94.4 | 88.9 |
| | | | Mat. 1/1 | | | | | | | | | |
| | | | Fet. 1/1; | 5,581 | 100.0 | 99.3 | 5,489 | 100.0 | 99.4 | 92 | 98.9 | 91.3 |
| | | | Mat. 1/1 | | | | | | | | | |
| MGB29 | 324X | 30 | Fet. 0/1; | 4,695 | 99.2 | 96.0 | 4,584 | 99.4 | 96.5 | 111 | 89.2 | 73.9 |
| | | | Mat. 0/0 | | | | | | | | | |
| | | | Fet. 0/0; | 4,467 | 99.3 | 94.5 | 4,360 | 99.5 | 95.1 | 107 | 90.7 | 71.0 |
| | | | Mat. 0/1 | | | | | | | | | |
| | | | Fet. 0/1; | 6,703 | 99.9 | 92.3 | 6,569 | 99.9 | 92.7 | 134 | 98.5 | 70.9 |
| | | | Mat. 0/1 | | | | | | | | | |
| | | | Fet. 1/1; | 2,350 | 99.7 | 90.6 | 2,305 | 99.8 | 90.9 | 45 | 97.8 | 75.6 |
| | | | Mat. 0/1 | | | | | | | | | |
| | | | Fet. 0/1; | 2,263 | 99.9 | 97.5 | 2,232 | 100.0 | 97.6 | 31 | 96.8 | 90.3 |
| | | | Mat. 1/1 | | | | | | | | | |
| | | | Fet. 1/1; | 5,846 | 99.9 | 99.3 | 5,753 | 99.9 | 99.5 | 93 | 97.9 | 89.3 |
| | | | Mat. 1/1 | | | | | | | | | |
| MGB42 | 211X | 30 | Fet. 0/1; | 4,919 | 99.3 | 95.9 | 4,814 | 99.5 | 96.2 | 105 | 91.4 | 81.0 |
| | | | Mat. 0/0 | | | | | | | | | |
| | | | Fet. 0/0; | 4,400 | 99.1 | 94.3 | 4,298 | 99.3 | 94.9 | 102 | 91.2 | 68.6 |
| | | | Mat. 0/1 | | | | | | | | | |
| | | | Fet. 0/1; | 6,566 | 99.8 | 86.4 | 6,441 | 99.8 | 86.7 | 125 | 98.4 | 71.2 |
| | | | Mat. 0/1 | | | | | | | | | |
| | | | Fet. 1/1; | 2,184 | 100.0 | 86.8 | 2,136 | 100.0 | 87.0 | 48 | 100.0 | 77.1 |
| | | | Mat. 0/1 | | | | | | | | | |
| | | | Fet. 0/1; | 2,294 | 100.0 | 97.6 | 2,254 | 100.0 | 97.9 | 40 | 97.5 | 80.0 |
| | | | Mat. 1/1 | | | | | | | | | |
| | | | Fet. 1/1; | 5,703 | 99.9 | 99.4 | 5,614 | 100.0 | 99.5 | 89 | 97.8 | 92.1 |
| | | | Mat. 1/1 | | | | | | | | | |
| MGB45 | 203X | 29 | Fet. 0/1; | 5,042 | 98.6 | 95.1 | 4,921 | 98.7 | 95.4 | 121 | 93.4 | 81.8 |
| | | | Mat. 0/0 | | | | | | | | | |
| | | | Fet. 0/0; | 5,584 | 99.3 | 94.7 | 5,455 | 99.5 | 95.2 | 129 | 90.7 | 71.3 |
| | | | Mat. 0/1 | | | | | | | | | |
| | | | Fet. 0/1; | 7,434 | 99.9 | 86.9 | 7,312 | 99.9 | 87.3 | 122 | 98.4 | 65.6 |
| | | | Mat. 0/1 | | | | | | | | | |
| | | | Fet. 1/1; | 2,399 | 99.8 | 89.4 | 2,346 | 99.9 | 89.7 | 53 | 96.2 | 75.5 |
| | | | Mat. 0/1 | | | | | | | | | |
| | | | Fet. 0/1; | 2,326 | 99.8 | 97.4 | 2,291 | 99.9 | 97.6 | 35 | 94.3 | 82.9 |
| | | | Mat. 1/1 | | | | | | | | | |
| | | | Fet. 1/1; | 5,425 | 99.9 | 99.4 | 5,347 | 99.9 | 99.5 | 78 | 97.4 | 89.7 |
| | | | Mat. 1/1 | | | | | | | | | |
| MGB26 | 105X | 25 | Fet. 0/1; | 4,678 | 98.6 | 95.6 | 4,573 | 98.8 | 96.0 | 105 | 91.4 | 79.1 |
| | | | Mat. 0/0 | | | | | | | | | |
| | | | Fet. 0/0; | 4,645 | 99.2 | 87.8 | 4,543 | 99.5 | 88.2 | 102 | 86.3 | 69.6 |
| | | | Mat. 0/1 | | | | | | | | | |
| | | | Fet. 0/1; | 6,583 | 99.7 | 73.3 | 6,457 | 99.8 | 73.7 | 126 | 95.2 | 51.6 |
| | | | Mat. 0/1 | | | | | | | | | |
| | | | Fet. 1/1; | 2,321 | 99.9 | 77.2 | 2,283 | 99.9 | 77.5 | 38 | 100.0 | 57.9 |
| | | | Mat. 0/1 | | | | | | | | | |
| | | | Fet. 0/1; | 2,387 | 99.9 | 96.2 | 2,342 | 100.0 | 96.5 | 45 | 95.6 | 82.2 |
| | | | Mat. 1/1 | | | | | | | | | |
| | | | Fet. 1/1; | 5,793 | 99.8 | 99.0 | 5,697 | 99.8 | 99.3 | 96 | 99.0 | 86.5 |
| | | | Mat. 1/1 | | | | | | | | | |
| MGB27 | 195X | 24 | Fet. 0/1; | 4,529 | 99.0 | 96.5 | 4,428 | 99.2 | 96.9 | 101 | 89.1 | 77.2 |
| | | | Mat. 0/0 | | | | | | | | | |
| | | | Fet. 0/0; | 4,702 | 99.5 | 94.1 | 4,601 | 99.7 | 94.5 | 101 | 92.1 | 76.2 |
| | | | Mat. 0/1 | | | | | | | | | |
| | | | Fet. 0/1; | 6,601 | 99.7 | 77.4 | 6,474 | 99.8 | 77.9 | 127 | 96.1 | 55.1 |
| | | | Mat. 0/1 | | | | | | | | | |
| | | | Fet. 1/1; | 2,253 | 99.7 | 83.0 | 2,215 | 99.9 | 83.7 | 38 | 92.1 | 44.7 |
| | | | Mat. 0/1 | | | | | | | | | |
| | | | Fet. 0/1; | 2,148 | 99.9 | 97.0 | 2,103 | 100.0 | 97.3 | 45 | 97.8 | 84.4 |
| | | | Mat. 1/1 | | | | | | | | | |
| | | | Fet. 1/1; | 5,870 | 99.9 | 99.4 | 5,774 | 99.9 | 99.5 | 96 | 99.0 | 95.8 |
| | | | Mat. 1/1 | | | | | | | | | |
| MGB38 | 279X | 22 | Fet. 0/1; | 4,543 | 99.3 | 96.9 | 4,444 | 99.4 | 97.4 | 99 | 95.0 | 77.8 |
| | | | Mat. 0/0 | | | | | | | | | |
| | | | Fet. 0/0; | 4,401 | 99.4 | 95.3 | 4,310 | 99.5 | 95.6 | 91 | 97.8 | 84.6 |
| | | | Mat. 0/1 | | | | | | | | | |
| | | | Fet. 0/1; | 6,307 | 99.8 | 79.8 | 6,206 | 99.9 | 80.1 | 101 | 98.0 | 59.4 |
| | | | Mat. 0/1 | | | | | | | | | |
| | | | Fet. 1/1; | 2,205 | 100.0 | 88.5 | 2,173 | 100.0 | 88.7 | 32 | 100.0 | 78.1 |
| | | | Mat. 0/1 | | | | | | | | | |
| | | | Fet. 0/1; | 2,164 | 100.0 | 98.8 | 2,121 | 100.0 | 99.1 | 43 | 97.7 | 83.7 |
| | | | Mat. 1/1 | | | | | | | | | |
| | | | Fet. 1/1; | 5,669 | 100.0 | 99.6 | 5,586 | 100.0 | 99.7 | 83 | 98.8 | 90.4 |
| | | | Mat. 1/1 | | | | | | | | | |
| MGB43 | 334X | 12 | Fet. 0/1; | 4,174 | 99.1 | 96.7 | 4,093 | 99.2 | 97.0 | 81 | 92.6 | 77.8 |
| | | | Mat. 0/0 | | | | | | | | | |
| | | | Fet. 0/0; | 5,268 | 99.2 | 86.4 | 5,142 | 99.4 | 86.8 | 126 | 89.7 | 70.6 |
| | | | Mat. 0/1 | | | | | | | | | |
| | | | Fet. 0/1; | 6,134 | 99.9 | 63.2 | 6,038 | 99.9 | 63.5 | 96 | 99.0 | 40.6 |
| | | | Mat. 0/1 | | | | | | | | | |
| | | | Fet. 1/1; | 2,103 | 100.0 | 61.5 | 2,069 | 100.0 | 61.7 | 34 | 100.0 | 52.9 |
| | | | Mat. 0/1 | | | | | | | | | |
| | | | Fet. 0/1; | 2,090 | 100.0 | 98.7 | 2,060 | 100.0 | 98.9 | 30 | 100.0 | 83.3 |
| | | | Mat. 1/1 | | | | | | | | | |
| | | | Fet. 1/1; | 5,456 | 100.0 | 99.5 | 5,388 | 100.0 | 99.7 | 68 | 98.5 | 85.3 |
| | | | Mat. 1/1 | | | | | | | | | |
| MGB41 | 217X | 9 | Fet. 0/1; | 4,738 | 94.3 | 92.0 | 4,619 | 94.7 | 92.8 | 119 | 79.8 | 62.2 |
| | | | Mat. 0/0 | | | | | | | | | |
| | | | Fet. 0/0; | 5,005 | 99.2 | 69.6 | 4,901 | 99.5 | 69.8 | 104 | 88.5 | 59.6 |
| | | | Mat. 0/1 | | | | | | | | | |
| | | | Fet. 0/1; | 6,796 | 99.8 | 65.0 | 6,685 | 99.9 | 65.3 | 111 | 96.4 | 47.7 |
| | | | Mat. 0/1 | | | | | | | | | |
| | | | Fet. 1/1; | 2,208 | 99.7 | 56.7 | 2,164 | 99.8 | 56.9 | 44 | 95.5 | 43.2 |
| | | | Mat. 0/1 | | | | | | | | | |
| | | | Fet. 0/1; | 2,493 | 99.8 | 91.1 | 2,442 | 100.0 | 91.4 | 51 | 98.0 | 80.4 |
| | | | Mat. 1/1 | | | | | | | | | |
| | | | Fet. 1/1; | 5,458 | 99.9 | 99.5 | 5,378 | 100.0 | 99.7 | 80 | 95.0 | 87.5 |
| | | | Mat. 1/1 | | | | | | | | | |

TABLE 10
Fetal Site Level Variant Detection

| | Mean | Fetal | After Filter NIFS Variant Detection (%)^{#} | | | | | | Unfiltered NIFS Variant Detection (%)* | | | | | |
|---|---|---|---|---|---|---|---|---|---|---|---|---|---|---|
| | Target | Frac. | SNV | | | Indel | | | SNV | | | Indel | | |
| Sample | Cov. | (%) | Count | Sens. | PPV | Count | Sens. | PPV | Count | Sens. | PPV | Count | Sens. | PPV |
| MGB22 | 269X | 40 | 22,996 | 98.3 | 95.4 | 534 | 85.1 | 75.8 | 27,234 | 99.7 | 80.6 | 1,550 | 93.4 | 25.8 |
| MGB20 | 161X | 39 | 22,942 | 98.1 | 95.3 | 538 | 87.0 | 80.4 | 27,508 | 99.6 | 79.5 | 1,099 | 95.1 | 39.3 |
| MGB31 | 232X | 32 | 23,252 | 98.3 | 95.4 | 527 | 87.0 | 74.5 | 28,043 | 99.7 | 79.1 | 1,260 | 92.4 | 30.6 |
| MGB29 | 323X | 30 | 22,761 | 98.5 | 95.4 | 562 | 86.8 | 73.9 | 27,394 | 99.7 | 79.2 | 1,585 | 93.8 | 25.9 |
| MGB42 | 211X | 30 | 22,812 | 98.6 | 94.6 | 556 | 89.2 | 74.3 | 27,582 | 99.7 | 78.2 | 1,489 | 94.8 | 27.5 |
| MGB45 | 203X | 29 | 23,413 | 98.2 | 95.8 | 538 | 88.6 | 77.7 | 28,626 | 99.5 | 78.3 | 1,241 | 96.1 | 33.6 |
| MGB26 | 104X | 25 | 22,428 | 98.0 | 96.0 | 515 | 86.3 | 80.5 | 26,387 | 99.4 | 81.6 | 1,011 | 95.8 | 41.1 |
| MGB27 | 195X | 24 | 22,227 | 98.3 | 95.1 | 522 | 86.7 | 78.9 | 27,633 | 99.6 | 76.5 | 1,257 | 93.9 | 32.5 |
| MGB38 | 279X | 22 | 22,173 | 98.6 | 95.2 | 514 | 87.3 | 73.7 | 26,408 | 99.7 | 79.9 | 1,326 | 95.4 | 28.6 |
| MGB43 | 334X | 12 | 20,647 | 98.9 | 95.2 | 450 | 85.1 | 69.1 | 24,649 | 99.7 | 79.7 | 1,617 | 93.6 | 19.1 |
| MGB41 | 217X | 9 | 22,659 | 97.7 | 95.3 | 503 | 84.2 | 81.0 | 27,112 | 98.6 | 79.5 | 1,186 | 92.2 | 33.6 |

| | | | | | | | | | | | | |
|---|---|---|---|---|---|---|---|---|---|---|---|---|
| Mean | 22,573 | 98.3 | 95.3 | 523 | 86.7 | 76.3 | 27,143 | 99.5 | 79.3 | 1,329 | 94.2 | 30.7 |
| Median | 22,761 | 98.3 | 95.3 | 527 | 86.8 | 75.8 | 27,394 | 99.7 | 79.5 | 1,260 | 93.9 | 30.6 |

^{#}Evaluates the presence or absence of all sites in the confirmation sample in the filtered list of sites detected by NIFS, without considering genotype. Sites which are maternal only (as determined by the confirmation sample and the maternal gDNA sample), and regions with less than 10x coverage in the confirmation sample, are excluded from the evaluation.
*Evaluates the presence or absence of all sites in the confirmation sample in the unfiltered list of sites detected by NIFS, without considering genotype. Sites which are maternal only (as determined by the confirmation sample and the maternal gDNA sample), and regions with less than 10x coverage in the confirmation sample, are excluded from the evaluation.
Bold indicates values highlighted in letter.

TABLE 11
Maternal Variant Detection and Genotyping Performance against Germline Maternal ES

| | | Mean | | Detection of Maternal Variants | | | | | | | Maternal Genotyping Performance* | | | |
|---|---|---|---|---|---|---|---|---|---|---|---|---|---|---|
| | | Target | Fetal | Variant Count | | | SNV (%) | | Indel (%) | | SNV (%) | | Indel (%) | |
| Sample | Trimester | Cov. | Frac. | TP | FP | FN | Sens. | PPV | Sens. | PPV | Sens. | PPV | Sens. | PPV |
| MGB20 | 3rd | 160.8 | 39 | 21,229 | 905 | 724 | 97.2 | 96.4 | 86.2 | 84.7 | 96.7 | 95.9 | 84.6 | 83.1 |
| MGB22 | 3rd | 268.7 | 40 | 20,883 | 908 | 850 | 97 | 96.7 | 82 | 84 | 96.1 | 95.8 | 79.6 | 81.2 |
| MGB26 | 3rd | 104.9 | 25 | 20,996 | 790 | 506 | 98.1 | 96.8 | 85.3 | 84.3 | 97.7 | 96.4 | 84.1 | 83.1 |
| MGB27 | 3rd | 195.4 | 24 | 20,964 | 817 | 368 | 98.5 | 96.4 | 87.9 | 82.8 | 98.3 | 96.3 | 86.9 | 81.9 |
| MGB28 | 3rd | 267.9 | 30 | 20,506 | 2,081 | 1,426 | 98 | 95.1 | 83.8 | 81.9 | 93.5 | 90.8 | 79.8 | 77.9 |
| MGB29 | 3rd | 323.6 | 30 | 20,938 | 850 | 425 | 98.4 | 96.4 | 84 | 84.2 | 98 | 96.1 | 83.7 | 84 |
| MGB30 | 3rd | 190 | 34 | 20,644 | 1,086 | 777 | 97.3 | 95.9 | 82.1 | 84.4 | 96.4 | 95 | 80.1 | 82.4 |
| MGB31 | 3rd | 231.6 | 32 | 22,006 | 861 | 578 | 97.8 | 96.6 | 85.8 | 86 | 97.4 | 96.2 | 84.2 | 84.4 |
| MGB32 | 3rd | 231.5 | 28 | 20,024 | 2,504 | 1,418 | 98.4 | 93.6 | 85.7 | 78.8 | 93.4 | 88.9 | 78.6 | 72.3 |
| MGB33 | 3 rd | 200.4 | 30 | 20,936 | 824 | 364 | 98.5 | 96.4 | 86.9 | 82.3 | 98.3 | 96.2 | 85.4 | 81 |
| MGB42 | 3rd | 211.4 | 30 | 20,497 | 888 | 363 | 98.5 | 96.1 | 85.4 | 81.9 | 98.3 | 95.9 | 84.9 | 81.5 |
| MGB44* | 3rd | 102.8 | 51 | 12,461 | 9,028 | 8,783 | 78.6 | 77.7 | 72.8 | 70.3 | 58.7 | 58 | 52.7 | 51 |
| MGB45 | 3rd | 203.3 | 29 | 22,490 | 828 | 469 | 98.2 | 96.7 | 85.1 | 82.1 | 98 | 96.5 | 83.9 | 81 |
| MGB47 | 3rd | 248.3 | 19 | 22,824 | 955 | 376 | 98.5 | 96.1 | 90.3 | 80.4 | 98.4 | 96 | 90.1 | 80.2 |
| MGB34 | 2nd | 158.8 | 13 | 21,760 | 939 | 259 | 98.9 | 95.9 | 88.8 | 85.5 | 98.8 | 95.9 | 88.6 | 85.3 |
| MGB35 | 2nd | 169.1 | 14 | 21,775 | 963 | 280 | 98.8 | 95.8 | 89.2 | 79.9 | 98.7 | 95.8 | 88.3 | 79.1 |
| MGB38 | 2nd | 279.2 | 22 | 20,240 | 977 | 288 | 98.7 | 95.5 | 90 | 83.2 | 98.6 | 95.4 | 88.9 | 82.2 |
| MGB40 | 2nd | 203.3 | 20 | 21,060 | 880 | 416 | 98.2 | 96.1 | 86.7 | 79.6 | 98.1 | 96 | 85.5 | 78.4 |
| MGB41 | 2nd | 216.8 | 9 | 21,390 | 955 | 311 | 98.8 | 95.9 | 89.4 | 81.1 | 98.6 | 95.7 | 89.1 | 80.8 |
| MGB43 | 2nd | 333.6 | 12 | 21,051 | 1,008 | 287 | 98.8 | 95.5 | 86.1 | 78.8 | 98.7 | 95.4 | 85.1 | 77.9 |
| MGB48 | 2nd | 174.2 | 6 | 21,026 | 724 | 535 | 97.6 | 96.7 | 83.5 | 85.9 | 97.5 | 96.7 | 82.4 | 84.7 |
| MGB49 | 2nd | 216 | 9 | 21,825 | 1,065 | 276 | 98.9 | 95.5 | 88.1 | 80.3 | 98.8 | 95.4 | 87.4 | 79.7 |
| MGB50 | 2nd | 250.7 | 16 | 20,949 | 900 | 357 | 98.5 | 96 | 87.6 | 81.6 | 98.3 | 95.9 | 87.1 | 81.1 |
| MGB36 | 1st | 131.9 | 14 | 21,184 | 810 | 260 | 98.9 | 96.4 | 88.9 | 87.4 | 98.8 | 96.3 | 87.9 | 86.3 |
| MGB37 | 1st | 205.8 | 8 | 20,933 | 924 | 306 | 98.7 | 95.9 | 87.8 | 79.3 | 98.6 | 95.8 | 87.6 | 79.1 |
| MGB39 | 1st | 221.9 | 8 | 20,956 | 955 | 238 | 99 | 95.8 | 87.4 | 78 | 98.9 | 95.6 | 87.4 | 78 |
| MGB46 | 1st | 295.3 | 10 | 20,716 | 919 | 352 | 98.5 | 95.9 | 91.1 | 81.5 | 98.3 | 95.8 | 90.6 | 81.1 |
| MGB51 | 1st | 262.2 | 14 | 20,576 | 968 | 499 | 98 | 95.8 | 85.4 | 77.3 | 97.6 | 95.5 | 84.9 | 76.9 |
| | | | Mean | 20,816 | 1,297 | 789 | 97.6 | 95.4 | 86.2 | 81.7 | 96.3 | 94.1 | 84.3 | 79.8 |
| | | | Median | 20,960 | 922 | 372 | 98.5 | 96.0 | 86.5 | 81.9 | 98.3 | 95.8 | 85.2 | 81.0 |
| | | | Sum | 582,839 | 36,312 | 22,091 | | | | | | | | |

*Maternal accuracy dramatically decreases when fetal fraction approaches 50% since the maternal and fetal unique are equivalent allele fractions. Genotype accuracy is calculated by comparing the maternal genotypes assigned by NIFS at each site to genotyping from the gDNA ES of the mother.

TABLE 12
Clinical Information for Samples

| | | | Genetic Testing | |
|---|---|---|---|---|
| Sample | Fetal | | beyond cfDNA | |
| ID | Sex | Fetal Anomaly | Aneuploidy Screen | Clinical Findings |
| MGB26* | XY | Bilateral hydronephrosis | No | NA |
| MGB38 | XY | Cleft lip/palate, eye | Microarray (detected 7q | Terminal Deletion |
| | | anomalies, possible brain | deletion) | on chr7 |
| | | anomaly | | |
| MGB39 | XY | Normal ultrasound, both | Targeted molecular | Homozgous for |
| | | parents carriers of cystic | testing for parental CF | pathogenic CFTR |
| | | fibrosis | variants | variant |
| MGB40 | XX | Normal ultrasound, both | Targeted molecular | Heterozygous |
| | | parents carriers of cystic | testing for parental CF | carrier for |
| | | fibrosis | variants | pathogenic CFTR |
| | | | | mutation |
| MGB41 | XY | Horseshoe kidney, single | Microarray (normal) and | None |
| | | umbilical artery | sgNIPT (Vistara) (low | |
| | | | risk) | |
| MGB42 | XY | Heterotaxy, cardiac | Microarray (normal) | VUS variant in |
| | | anomalies | | ZIC3 |
| MGB43 | XX/XX | Monochorionic-diamniotic | Microarray (normal x2); | None |
| | | twins; twin A: renal | research exome sent on | |
| | | anomaly; twin B: congenital | twin B | |
| | | diaphragmatic hernia, growth | | |
| | | restriction | | |
| MGB44 | XY | Omphalocele, ectopia cordis, | Microarray (normal); | None |
| | | pulmonary stenosis, hydrops | exome sequencing | |
| | | | (negative) | |
| MGB45 | XY | Suspected aortic coarctation | Microarray (normal) | None |
| MGB46 | XY | Increased nuchal | sgNIPT (Vistara) (low | None |
| | | translucency | risk); declined CVS as | |
| | | | NT normalized in the 1st | |
| | | | trimester | |
| MGB47 | XY | Micrognathia | Microarray (normal); | Splicing variant in |
| | | | Stickler syndrome panel | COL2A1 |
| | | | molecular testing, | |
| | | | positive for COL2A1 | |
| | | | pathogenic variant | |
| MGB48 | XY | Cerebral ventriculomegaly | Microarray (normal) and | None |
| | | | sgNIPT (Vistara) (low | |
| | | | risk) | |
| MGB49 | XX | Positive aneuploidy screen | Microarray (normal on | Vanishing Twin |
| | | | ongoing pregnancy) | |
| MGB50 | XY | Cerebral ventriculomegaly | Microarray (normal) and | None |
| | | | sgNIPT (Vistara) (low | |
| | | | risk) | |
| MGB51 | XY | Increased nuchal | Microarray (normal) and | None |
| | | translucency | sgNIPT (Vistara) (low | |
| | | | risk) | |

*Excluded from clinical assessment because the patient never received follow up testing

TABLE 13
Clinically Relevant Variants

| | Diagnostic Variants (n = 4) | | | | Other Variants of Interest (n = 3) | | |
|---|---|---|---|---|---|---|---|
| Chr | chr7 | chr7 | chr12 | chrY^{#} | chrX | chr7 | chr13 |
| Position | 117559590 | 155368937- | 47982610 | 1-57227415 | 137568993 | 117559590 | 32340300 |
| (hg38) | | 159327017^{&} | | | | | |
| Protein | p.F508del | — | c.2194-1G > A | — | p.P387_K395del | p.F508del | p.S1982Rfs22 |
| Change | | | | | | | |
| Gene(s) | CFTR | — | COL2A1 | — | ZIC3 | CFTR | BRCA2 |
| Disease | Cystic | Terminal 4 | Stickler | Abnormal | Heterotaxy | Cystic | Susceptibility |
| Description | Fibrosis | MB | Syndrome | Aneuploidy | | Fibrosis | to Cancer |
| | | Deletion | | Test | | (CF) | |
| Inheritance | AR | AD | AD | | XLR | AR | AD |
| Mode of | | | | | | | |
| Disease | | | | | | | |
| Fetal | 1/1 | 0/1 | 0/1 | — | 0/1 | 0/1 | 0/1 |
| Genotype | | | | | | | |
| Maternal | 0/1 | 0/0 | 0/0 | — | 0/1 | 0/1 | 0/0 |
| Genotype | | | | | | | |
| Paternal | 0/1 | 0/0 | unknown | — | unknown | 0/1 | 0/1 |
| Genotype* | | | | | | | |
| Clinical | High risk | Fetus | Fetus | Result | VUS in Male | Maternal, | Increased |
| Interpretation | for CF | Affected | Affected | caused | Fetus | Paternal, | Risk for |
| | | | | by | | & Fetal | Cancer |
| | | | | vanishing | | Carrier | |
| | | | | male twin | | | |
| Clinically | Yes | Yes | Yes | Yes | Yes | Yes | No |
| Validated | | | | | | | |
| ID | MGB39 | MGB38 | MGB47 | MGB49 | MGB42 | MGB40 | MGB50 |
| Fetal Sex | XY | XY | XY | XX | XY | XX | XY |

*Paternal genotypes derived from separately collected DNA that underwent exome sequencing; see Table 1
^{#}Confirmation of a vanishing twin was detected by NIFS during sex inference; see FIG. 4
^{&}Note that these breakpoints are the minimal breakpoints as defined by identified deleted exons

TABLE 14
Maternal Carrier Variants

| | Position | Protein | | |
|---|---|---|---|---|
| Chr | (hg38) | Change | Gene | Disease Description |
| chr1 | 150553749 | p.Q256Pfs*38 | ADAMTSL4 | Ectopia Lentis et Pupillae |
| chr1 | 169549811 | p.R534Q | F5 | Factor V Deficiency |
| chr1 | 216247094 | p.E767Sfs*21 | USH2A | Usher Syndrome Type IIA |
| chr2 | 44312653 | p.M467T | SLC3A1 | Cystinuria |
| chr3 | 50345495 | p.S29P | ZMYND10 | Primary Ciliary Dyskinesia |
| chr4 | 67740682 | p.R262Q | GNRHR | Hypogonadotropic hypogonadism 7 |
| | | | | without anosmia |
| chr4 | 121854790 | p.T2111 | BBS7 | Bardet-Biedl syndrome |
| chr4 | 122927721 | p.R83Q | SPATA5 | Neurodevelopmental disorder with |
| | | | | hearing loss, seizures, and brain |
| | | | | abnormalities |
| chr5 | 148086434 | p.D106Wfs*7 | SPINK5 | Netherton syndrome |
| chr7 | 74783529 | p.W193X | NCF1 | Chronic granulomatous disease 1 |
| chr7 | 117559590 | p.F508del | CFTR | Cystic Fibrosis |
| chr7 | 117559590 | p.F508del | CFTR | Cystic Fibrosis |
| chr7 | 117559590 | p.F508del | CFTR | Cystic Fibrosis |
| chr7 | 117559590 | p.F508del | CFTR | Cystic Fibrosis |
| chr8 | 31141504 | p.W1014X | WRN | Werner Syndrome |
| chr8 | 142912806 | c.1200 + 1G > A | CYP11B2 | Corticosterone Methyloxidase Type |
| | | | | I Deficiency |
| chr10 | 13112464 | p.D128Rfs22 | OPTN | Glaucoma |
| chr11 | 5227002 | p.E7V | HBB | Sickle Cell Anemia |
| chr11 | 59845374 | c.79 + 1G > A | CBLIF | Intrinsic Factor Deficiency |
| chr11 | 71491856 | p.A573T | NADSYN1 | Vertebral, cardiac, renal, and limb |
| | | | | defects syndrome 3 |
| chr12 | 6034812 | p.R854Q | VWF | von Willebrand disease |
| chr12 | 57244322 | p.W98S | STAC3 | Congenital myopathy 13 |
| chr12 | 102866632 | p.R158Q | PAH | Phenylketonuria |
| chr12 | 110619957 | c.173 + 1G > A | TCTN1 | Joubert syndrome 13 |
| chr13 | 20189413 | p.Q57X | GJB2 | Deafness |
| chr13 | 20189481 | p.M34T | GJB2 | Deafness, autosomal recessive 1A |
| chr13 | 20189546 | p.G12Vfs2 | GJB2 | Deafness |
| chr13 | 51944145 | p.H862Q | ATP7B | Wilson disease |
| chr13 | 51950132 | p.G707R | ATP7B | Wilson Disease |
| chr15 | 71813573 | p.R311Q | NR2E3 | Enhanced S-cone syndrome |
| chr15 | 89321792 | p.G848S | POLG | Mitochondrial DNA Depletion |
| | | | | Syndrome |
| chr16 | 3243310 | p.V726A | MEFV | Familial Mediterranian Fever |
| chr17 | 18154189 | p.Q2716R | MYO15A | Deafness, autosomal recessive 3 |
| chr17 | 50167653 | p.R77C | SGCA | Muscular dystrophy, limb-girdle, |
| | | | | autosomal recessive 3 |
| chr17 | 80214757 | p.G122R | SGSH | Mucopolysaccharidosis type IIIA |
| chr19 | 12896249 | p.R227P | GCDH | Glutaric Acidemia I |
| chr19 | 38502902 | p.Q2620X | RYR1 | Central Core Disease |

Note that 16/28 (57.1%) samples had at least one maternal carrier variant.

### REFERENCES

1. Lowther C, Valkanas E, Giordano J L, et al. Systematic evaluation of genome sequencing for the assessment of fetal structural anomalies [Internet]. bioRxiv. 2020; Available from: biorxiv.org/content/10.1101/2020.08.12.248526.abstract
2. Talkowski M E, Ordulu Z, Pillalamarri V, et al. Clinical diagnosis by whole-genome sequencing of a prenatal sample. N Engl J Med 2012; 367(23):2226-32.
3. Tolusso L K, Hazelton P, Wong B, Swarr D T. Beyond diagnostic yield: prenatal exome sequencing results in maternal, neonatal, and familial clinical management changes. Genet Med 2021; 23(5):909-17.
4. Gregg A R, Skotko B G, Benkendorf J L, et al. Noninvasive prenatal screening for fetal aneuploidy, 2016 update: a position statement of the American College of Medical Genetics and Genomics. Genet Med 2016; 18(10):1056-65.
5. American College of Obstetricians and Gynecologists' Committee on Practice Bulletins-Obstetrics, Committee on Genetics, Society for Maternal-Fetal Medicine. Screening for Fetal Chromosomal Abnormalities: ACOG Practice Bulletin, Number 226. Obstet Gynecol 2020; 136(4):e48-69.
6. Bianchi D W, Parker R L, Wentworth J, et al. DNA sequencing versus standard prenatal aneuploidy screening. N Engl J Med 2014; 370(9):799-808.
7. Norton M E, Jacobsson B, Swamy G K, et al. Cell-free DNA analysis for noninvasive examination of trisomy. N Engl J Med 2015; 372(17):1589-97.
8. Yatsenko S A, Peters D G, Saller D N, Chu T, Clemens M, Rajkovic A. Maternal cell-free DNA-based screening for fetal microdeletion and the importance of careful diagnostic follow-up. Genet Med 2015; 17(10):836-8.
9. Zhang J, Li J, Saucier J B, et al. Non-invasive prenatal sequencing for multiple Mendelian monogenic disorders using circulating cell-free fetal DNA. Nat Med 2019; 25(3):439-47.
10. Breveglieri G, D'Aversa E, Finotti A, Borgatti M. Non-invasive Prenatal Testing Using Fetal DNA. Mol Diagn Ther 2019; 23(2):291-9.
11. Dungan J S, Klugman S, Darilek S, et al. Noninvasive prenatal screening (NIPS) for fetal chromosome abnormalities in a general-risk population: An evidence-based clinical guideline of the American College of Medical Genetics and Genomics (ACMG). Genet Med 2023; 25(2):100336.
12. Rose N C, Barrie E S, Malinowski J, et al. Systematic evidence-based review: The application of noninvasive prenatal screening using cell-free DNA in general-risk pregnancies. Genet Med 2022; 24(7):1379-91.
13. Fan H C, Gu W, Wang J, Blumenfeld Y J, El-Sayed Y Y, Quake S R. Non-invasive prenatal measurement of the fetal genome. Nature 2012; 487(7407):320-4.
14. Provenzano A, Farina A, Seidenari A, et al. Prenatal Noninvasive Trio-WES in a Case of Pregnancy-Related Liver Disorder. Diagnostics (Basel) [Internet]2021; 11(10). Available from: dx.doi.org/10.3390/diagnosticsl 1101904
15. Filer D L, Mieczkowski P A, Brandt A, et al. Noninvasive prenatal exome sequencing diagnostic utility limited by sequencing depth and fetal fraction. Prenat Diagn [Internet]2021; Available from: dx.doi.org/10.1002/pd.6009
16. Guo M H, Gregg A R. Estimating yields of prenatal carrier screening and implications for design of expanded carrier screening panels. Genet Med 2019; 21(9):1940-7.
17. Ben-Shachar R, Svenson A, Goldberg J D, Muzzey D. A data-driven evaluation of the size and content of expanded carrier screening panels. Genet Med 2019; 21(9):1931-9.
18. Saunders C J, Miller N A, Soden S E, et al. Rapid whole-genome sequencing for genetic disease diagnosis in neonatal intensive care units. Sci Transl Med 2012; 4(154):154ra135.
19. Balloux F, Bronstad Brynildsrud O, van Dorp L, et al. From Theory to Practice: Translating Whole-Genome Sequencing (WGS) into the Clinic. Trends Microbiol 2018; 26(12):1035-48.
20. Miller D T, Lee K, Abul-Husn N S, et al. ACMG S F v3.1 list for reporting of secondary findings in clinical exome and genome sequencing: A policy statement of the American College of Medical Genetics and Genomics (ACMG). Genet Med 2022; 24(7):1407-14.
21. Monaghan K G, Leach N T, Pekarek D, Prasad P, Rose N C, ACMG Professional Practice and Guidelines Committee. The use of fetal exome sequencing in prenatal diagnosis: a points to consider document of the American College of Medical Genetics and Genomics (ACMG). Genet Med 2020; 22(4):675-80.
22. Van den Veyver I B, Chandler N, Wilkins-Haug L E, Wapner R J, Chitty L S, ISPD Board of Directors. International Society for Prenatal Diagnosis Updated Position Statement on the use of genome-wide sequencing for prenatal diagnosis. Prenat Diagn 2022; 42(6):796-803.
23. McElrath T F, Lim K-H, Pare E, et al. Longitudinal evaluation of predictive value for preeclampsia of circulating angiogenic factors through pregnancy. Am J Obstet Gynecol 2012; 207(5):407.e1-7.
24. Li H. Aligning sequence reads, clone sequences and assembly contigs with BWA-MEM [Internet]. arXiv [q-bio.GN]. 2013; Available from: arxiv.org/abs/1303.3997
25. Poplin R, Ruano-Rubio V, DePristo M A, et al. Scaling accurate genetic variant discovery to tens of thousands of samples [Internet]. bioRxiv. 2018 [cited 2019 Nov. 21]; 201178. Available from: biorxiv.org/content/10.1101/201178v3.abstract
26. O'Leary N A, Wright M W, Brister J R, et al. Reference sequence (RefSeq) database at NCBI: current status, taxonomic expansion, and functional annotation. Nucleic Acids Res 2016; 44(D1):D733-45.
27. Benjamin D, Sato T, Cibulskis K, Getz G, Stewart C, Lichtenstein L. Calling Somatic SNVs and Indels with Mutect2 [Internet]. bioRxiv. 2019 [cited 2022 Apr. 12]; 861054. Available from: biorxiv.org/content/10.1101/861054v1
28. Karczewski K J, Francioli L C, Tiao G, et al. The mutational constraint spectrum quantified from variation in 141,456 humans. Nature 2020; 581(7809):434-43.
29. Bekker J, Davis J. Learning from positive and unlabeled data: a survey. Mach Learn 2020; 109(4):719-60.
30. Li H. Toward better understanding of artifacts in variant calling from high-coverage samples. Bioinformatics 2014; 30(20):2843-51.
31. Bingham E, Chen J P, Jankowiak M, et al. Pyro: Deep Universal Probabilistic Programming. J Mach Learn Res 2019; 20(28):1-6.
32. Fu J M, Satterstrom F K, Peng M, et al. Rare coding variation provides insight into the genetic architecture and phenotypic context of autism. Nat Genet 2022; 54(9):1320-31.
33. Cleary J G, Braithwaite R, Gaastra K, et al. Joint variant and de novo mutation identification on pedigrees from high-throughput sequencing data. J Comput Biol 2014; 21(6):405-19.
34. Cleary J G, Braithwaite R, Gaastra K, et al. Comparing Variant Call Files for Performance Benchmarking of Next-Generation Sequencing Variant Calling Pipelines [Internet]. bioRxiv. 2015 [cited 2023 Jun. 15]; 023754. Available from: biorxiv.org/content/10.1101/023754v2
35. Manichaikul A, Mychaleckyj J C, Rich S S, Daly K, Sale M, Chen W-M. Robust relationship inference in genome-wide association studies. Bioinformatics 2010; 26(22):2867-73.
36. Danecek P, Bonfield J K, Liddle J, et al. Twelve years of SAMtools and BCFtools. Gigascience [Internet]2021; 10(2). Available from: dx.doi.org/10.1093/gigascience/giab008
37. Wang K, Li M, Hakonarson H. ANNOVAR: functional annotation of genetic variants from high-throughput sequencing data. Nucleic Acids Res 2010; 38(16):e164.
38. Ioannidis N M, Rothstein J H, Pejaver V, et al. REVEL: An Ensemble Method for Predicting the Pathogenicity of Rare Missense Variants. Am J Hum Genet 2016; 99(4):877-85.
39. Landrum M J, Lee J M, Benson M, et al. ClinVar: improving access to variant interpretations and supporting evidence. Nucleic Acids Res 2018; 46(D1):D1062-7.
40. Amberger J S, Bocchini C A, Schiettecatte F, Scott A F, Hamosh A. Omim.org: Online Mendelian Inheritance in Man (OMIM®), an online catalog of human genes and genetic disorders. Nucleic Acids Res 2015; 43(Database issue):D789-98.
41. Jaganathan K, Kyriazopoulou Panagiotopoulou S, McRae J F, et al. Predicting Splicing from Primary Sequence with Deep Learning. Cell 2019; 176(3):535-548.e24.
42. Gregg A R, Aarabi M, Klugman S, et al. Screening for autosomal recessive and X-linked conditions during pregnancy and preconception: a practice resource of the American College of Medical Genetics and Genomics (ACMG). Genet Med 2021; 23(10):1793-806.
43. Harrison S M, Biesecker L G, Rehm H L. Overview of Specifications to the ACMG/AMP Variant Interpretation Guidelines. Curr Protoc Hum Genet 2019; 103(1):e93.
44. Richards S, Aziz N, Bale S, et al. Standards and guidelines for the interpretation of sequence variants: a joint consensus recommendation of the American College of Medical Genetics and Genomics and the Association for Molecular Pathology. Genet Med 2015; 17(5):405-24.
45. Riggs E R, Andersen E F, Cherry A M, et al. Technical standards for the interpretation and reporting of constitutional copy-number variants: a joint consensus recommendation of the American College of Medical Genetics and Genomics (ACMG) and the Clinical Genome Resource (ClinGen). Genet Med 2020; 22(2):245-57.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A computer-implemented method for assigning maternal or fetal origin to one or more genetic variants in cell free DNA (cfDNA) from a sample from a pregnant mammal, preferably a pregnant human, the method comprising:
(a) accessing, from memory, a probabilistic model for assigning maternal or fetal origin to genetic variants in DNA from a sample obtained from a pregnant mammal, wherein the model assigns maternal or fetal origin based on a combination of fetal fraction and/or DNA fragment size and other sequencing features;
(b) inputting, into the model, a set of values representing one or more genetic variants detected in the cfDNA from a peripheral blood sample from a pregnant mammal, wherein the values include empirically determined sequence information, e.g., ratio of different bases in the read, and DNA fragment size information, e.g, a rank sum statistic, for each genetic variant; and
(c) assigning, using the model, maternal or fetal origin for the one or more genetic variants.

2. The method of claim 1, wherein the genetic variants comprise single nucleotide variants (SNVs), indels, and/or copy number variations (CNVs).

3. The method of claim 1, wherein an initial set of values representing the one or more genetic variants is obtained by a method comprising:
aligning raw sequencing reads derived from the cfDNA to a reference genome sequence;
transforming the raw sequencing reads into consensus reads; realigning the consensus reads to the reference genome sequence, thereby producing a set of aligned consensus reads;
identifying consensus reads that differ from the reference genome;
assigning consensus reads that differ from the reference genome as alternate alleles and assigning consensus reads that match the reference genome as reference alleles, and determining a fragment size rank sum statistic representing the distribution of the estimated fragment sizes of reads supporting the reference allele as compared to the distribution of the fragment sizes of reads supporting the alternate allele,
thereby obtaining an initial set of values representing sequence identity and DNA fragment size rank sum statistic for one or more genetic variants.

4. The method of claim 3, wherein each of the raw sequencing reads comprises a unique molecular identifier (UMI); and the method comprises transforming the raw sequencing reads into a single consensus read for each UMI.

5. The method of claim 1, further comprising selecting a set of candidate variants before step (b), by a method comprising:
accessing, from memory, a machine learning classifier, optionally a random forest based model, wherein the machine learning classifier is trained using a set of predetermined filter criteria and a subset of sites present in the sample or in reference samples to identify potential false positive (FP) sites;
inputting, into the machine learning classifier, the initial set of variants; and
filtering, using the trained machine learning classifier to remove a set of variants enriched for false positive (FP) sites, thereby selecting a set of candidate variants from the initial set.

6. The method of claim 1, wherein the probabilistic model is a Bayesian Mixture Model that simultaneously estimates fetal fraction and assigns fetal or maternal origin for each variant site in the set.

7. The method of claim 6, wherein the Bayesian Mixture Model is a Bayesian Gaussian Mixture Model constrained over variant allele fraction and fragment size rank sum statistic.

8. The method of claim 6, wherein the fetal fraction of the sample is modeled as a latent variable (f) and mean of the variant allele fraction distribution is set for each component based on f

9. The method of claim 6, wherein the fetal fraction is estimated based on a reference fetal fraction determined based on clusters derived from VAF across sites.

10. The method of claim 1, further comprising outputting a list of one or more genetic variants identified as having fetal origin and/or one or more genetic variants identified as having maternal origin.

11. The method of claim 1, further comprising: comparing the genetic variants to a database that comprises a list of genetic variants and information regarding variants that are potentially medically relevant to the fetus or mother;
identifying variants present in the fetus or the mother that are potentially medically relevant; and
outputting a list of the one or more genetic variants identified as having fetal origin and/or one or more genetic variants identified as having maternal origin that potentially medically relevant.

12. The method of claim 11, wherein the methods further comprise the methods can further include recommending further testing based on the presence of variants that are potentially medically relevant.

13. The method of claim 12, wherein the further testing comprises amniocentesis or chorionic villus sampling (CVS); further monitoring of the fetus via ultrasonography; or genetic testing of the mother.

14. The method of claim 1, further comprising using high throughput sequencing on cfDNA extracted from a single sample of peripheral blood from the mother, optionally wherein exome capture is performed before the sequencing.

15. The method of claim 14, wherein adaptors with common PCR primer sequences and unique molecular identifiers (UMIs) are attached to the cfDNA, and PCR amplification is performed before the sequencing.

16. The method of claim 14, further comprising enriching the sample for fetal DNA, optionally by contacting the cfDNA with a plurality of oligonucleotides that bind to portions of the fetal genome, optionally comprising fetal protein-coding genes or other regions of the fetal genome that may be relevant to clinical interpretation or variant identification.
